(19) United States
(12) Patent Application Publication
(10) Pub. No.: US 2023/0159552 A1
(43) Pub. Date: May 25, 2023

(54) MEAYAMYCIN AND ITS ANALOGUES, METHODS FOR THEIR PREPARATION, AND METHODS OF USE

(71) Applicants: BRISTOL-MYERS SQUIBB COMPANY, Princeton, NJ (US); THE SCRIPPS RESEARCH INSTITUTE, La Jolla, CA (US)

(72) Inventors: Dale L. BOGER, La Jolla, CA (US); Naidu S. CHOWDARI, Dublin, CA (US); Sanjeev GANGWAR, Foster City, CA (US)

(21) Appl. No.: 17/916,947

(22) PCT Filed: Apr. 8, 2021
(86) PCT No.: PCT/US2021/026408

§ 371 Date: Oct. 4, 2022

Related U.S. Application Data
(60) Provisional application No. 63/007,564, filed on Apr. 9, 2020.

(51) Int. Cl. C07D 493/10; A61K 31/35

(52) U.S. Cl. CPC C07D 493/10; A61K 31/35

(57) ABSTRACT

Compounds according to formula (I), where R is as defined herein, have anti-cancer properties.

### CROSS-REFERENCE TO RELATED APPLICATIONS

This application claims the benefit under 35 U.S.C. §119(e) of US Provisional Application Ser. No. 63/007564, filed Apr. 9, 2020; the disclosure of which is incorporated herein by reference in its entirety for all purposes.

### STATEMENT REGARDING FEDERALLY SPONSORED RESEARCH

This invention was made in part with US Government support under Grant No. CA042056 D.L.B, awarded by National Institutes of Health. The US Government has certain rights in this invention.

### BACKGROUND OF THE DISCLOSURE

This disclosure relates to meayamycin and analogues thereof having cytotoxic activity, methods for their preparation, and methods of using them, particularly as anti-cancer agents.

FR901464, originally isolated from the bacteria Pseudomonas sp. No. 2663 (Ref. 1), is the first member of a growing class of potent antitumor antibiotics that now includes the spliceostatins (Ref. 2) and thailanstatins (Ref. 3). Total syntheses of FR901464 confirmed the assigned structure and relative stereochemistry within each noncontiguous subunit and permitted assignment of its absolute stereochemistry (Refs. 4-8).

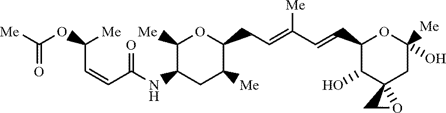 [SMILES: CC(=O)O[C@@H](C)/C=C\C(=O)N[C@@H]1C[C@H](C)[C@H](C/C=C(C)/C=C/[C@H]2O[C@](C)(O)C[C@@]3(CO3)[C@@H]2O)O[C@@H]1C]

Koide and colleagues investigated the limiting instability of FR901464 under physiological conditions and confirmed earlier observations that the right-hand subunit was responsible. They found that replacement of the tertiary alcohol with a methyl group not only avoided the rapid compound degradation but also improved potency as much as 100-fold (Refs. 4, 7). The resulting synthetic analogues, named the meayamycins, represented an early and key contribution to the growing collection of analogues in the class accessible by total synthesis (Refs. 4, 6, 9, 13, 17). Some representative meayamycin structures are shown following.

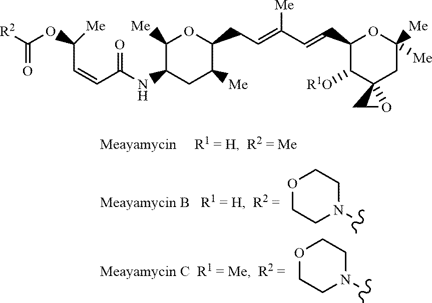

Yoshida disclosed that FR901464 binds a subunit of the spliceosome and acts through a new mechanism of action: inhibition of the action of the spliceosome complex by disrupting conversion of pre-messenger RNA (pre-mRNA) to mRNA (Ref. 14). Shortly thereafter, Koide and colleagues established that the meayamycins act through an analogous mechanism of action (Ref. 15). Spliceosome inhibition has been shown to be effective in controlling both cancer cell proliferation and metastasis, with selected members of the class exhibiting efficacious in vivo antitumor activity (Ref. 16).

### BRIEF SUMMARY OF THE DISCLOSURE

This disclosure relates to the synthesis of meayamycin and novel analogues thereof, especially modifications centered on left-hand subunit, and includes a short, scalable total synthesis of meayamycin proper.

In one aspect, this disclosure provides a compound according to formula (I):

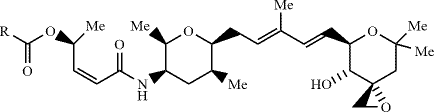

wherein R is

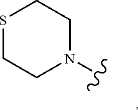

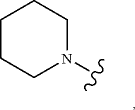

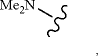

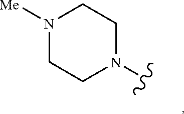

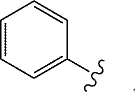

or

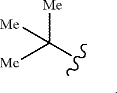

In another aspect, there is provided a method of treating a subject suffering cancer, comprising administering to such subject a therapeutically effective amount of a compound according to formula (I), in particular leukemia, colon cancer, and breast cancer.

### DETAILED DESCRIPTION OF THE DISCLOSURE

### Synthetic Strategy

Each of the three subunits (7, 15, and 22) used to assemble meayamycin (1) or its analogs was derived from chiral pool starting intermediates such that all 8 chiral centers are introduced or controlled by chiral centers found in readily available, inexpensive starting materials, as shown in the Retrosynthetic Analysis below.

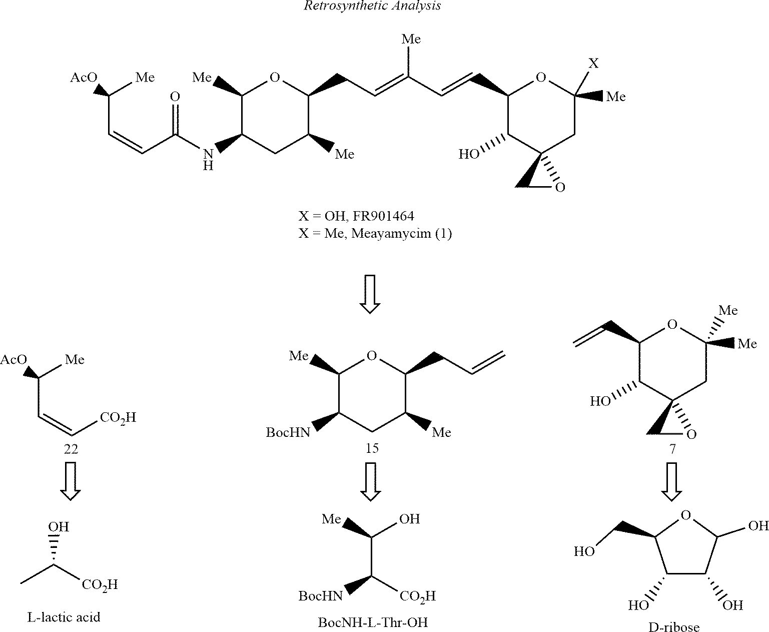

The most challenging of the three subunits was the right-hand tetrahydropyran 7, bearing three of the stereocenters and the essential reactive epoxide. Referring to Scheme 1 below, 7 was accessed in four steps from the known aldehyde 2 (Ref. 18), itself prepared in three straightforward steps from D-ribose. Grignard addition of commercially available 2-methylallylmagnesium chloride (2 equiv) to aldehyde 2 (THF, 5 h, 0-25° C., 5 h, 61-65%) provided 3 as an inconsequential mixture of diastereomers. Dess-Martin periodinane (DMP) oxidation of alcohol 3 (1.5 equiv DMP, CH₂Cl₂, 0° C., 3 h, 78-88%) and subsequent acid-catalyzed acetonide deprotection, alkene isomerization into conjugation with the ketone, and 6-endo-trig cyclization of the liberated distal alcohol provided cyclic ketone 6. Initial optimization of this reaction found that treatment of 3 with pyridinium p-toluenesulfonate (PPTS) in MeOH (0.2 equiv of PPTS, 60° C., 4 h) afforded principally the corresponding diol and subsequent addition of aq. 1 M HCl (MeOH/H₂O 9:1, 60° C., 2-4 h) effected the isomerization of the double bond into conjugation with the ketone to provide compound 5. Without purification, treatment of crude compound 5 with Amberlyst-15 (CHCl₃, 12 h, 80° C., 12 h) then effected the distal alkoxy 6-endo-trig conjugate addition to provide compound 6 (76% overall). Final conversion of compound 6 to the right-hand subunit 7 was achieved by diastereoselective epoxide introduction (61-73%) following the protocol of Ref. 19. In addition to the concise nature of the synthesis of 7 from a commercially available inexpensive starting material (4-5 steps, 30% overall from 2; 7-8 steps, 20% overall from D-ribose), the approach of Scheme 2 avoids the generation of diastereomers and provides full control of the absolute stereochemistry.

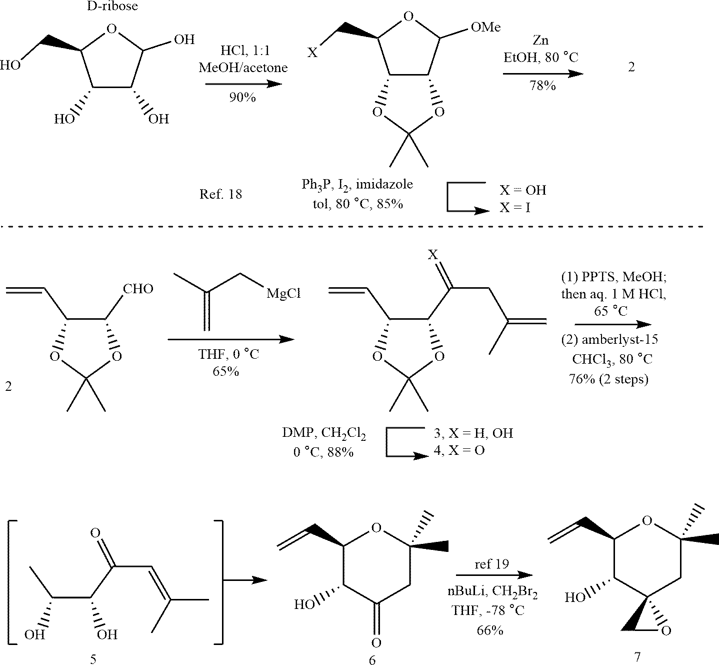

The central tetrahydropyran 15, bearing four chiral centers, was also accessed by an approach (Scheme 2) that relied on the chiral pool to set the relative and absolute stereochemistry and was based in part on the method of Ref. 6. Central to the synthesis of 15 was the use of the known starting material 9 (Ref. 20), the BocNH—L—Thr derived variant of Garner’s aldehyde, available in three steps from BocNH—L—Thr (1 equiv MeONHMe, 1.2 equiv EDCI, 1.2 equiv HOBt, 2 equiv (iPr₂)NEt, CH₂CI₂, 25° C., 22 h; 0.2 equiv PPTS, 10 equiv MeC(OMe)₂Me, THF, reflux, 18 h, 88% for two steps) including the reported DIBAL-H reduction of the Weinreb amide (2 equiv DIBAL-H, CH₂Cl₂, -78° C., 3 h) (Ref. 20). A subsequent Z-selective modified Wadsworth-Horner-Emmons reaction of 10 (Ref. 21) with the crude aldehyde 9 provided the α,β-unsaturated ester 11 (86%, 4.6:1 Z:E) where the preferential generation of the Z-isomer facilitates but may not be required for an ensuing lactonization. It was found that acid-catalyzed N,O-ketal cleavage effected with 10-camphorsulfonic acid (CSA) and in situ lactonization (0.05 equiv CSA, MeOH, 23° C., 4 d, 74%) provided 12 in a single step. This same intermediate was utilized in the Koide synthesis of 15 but was prepared in a longer, technically more challenging synthetic sequence. The subsequent alkene reduction of 12 proceeded with a lower diastereoselectivity (6:1 vs 10:1) and provided 13 contaminated with a minor amount of lactone ethanolysis product in our hands under conditions reported (2 mol % PtO₂, H₂, EtOH, 23° C., 2 h, 98%). (Ref. 6). For our purposes, this was reoptimized to provide 13 in high yield (quantitative) and excellent diastereoselectivity (10:1, THF > EtOH, iPrOH, EtOAc) simply through use of THF as solvent (23° C., 15 h) without minor competitive lactone ring opening by solvent. More substantial optimizations were required for our implementation of the reported two-step conversion of 13 to 15. (Ref. 6). Significant amounts of double addition product were observed when the reaction of commercially available allylmagnesium chloride (1.9 equiv) with 13 was conducted as detailed in Ref. 6, likely accounting for the lower overall conversion of 13 to 15 than reported herein. Although a reduction in the number of equivalents of allylmagnesium chloride (1.3-1.6 equiv) attenuated the over addition reaction, increasing amounts of recovered starting 13 offset any improvement in this selectivity. However, by lowering the reaction temperature (-98 vs -78° C.) and adjusting the reaction solvent (2-MeTHF vs THF), 14 was obtained in excellent yield (87%) with minimal amounts of over addition (6%) or recovered starting lactone (7%). Final diastereoselective reduction of the lactol provided 15 in improved conversions (49%) provided the triethylsilane (10 equiv) and trifluoroethanol (TFE, 8 equiv) were also added at -78° C. and stirred for 10 min prior to addition of BF₃-OEt₂ (4 equiv) and was conducted on gram scales, completing the synthesis of the central subunit (25% overall, 8 steps from BocNH-L-Thr).

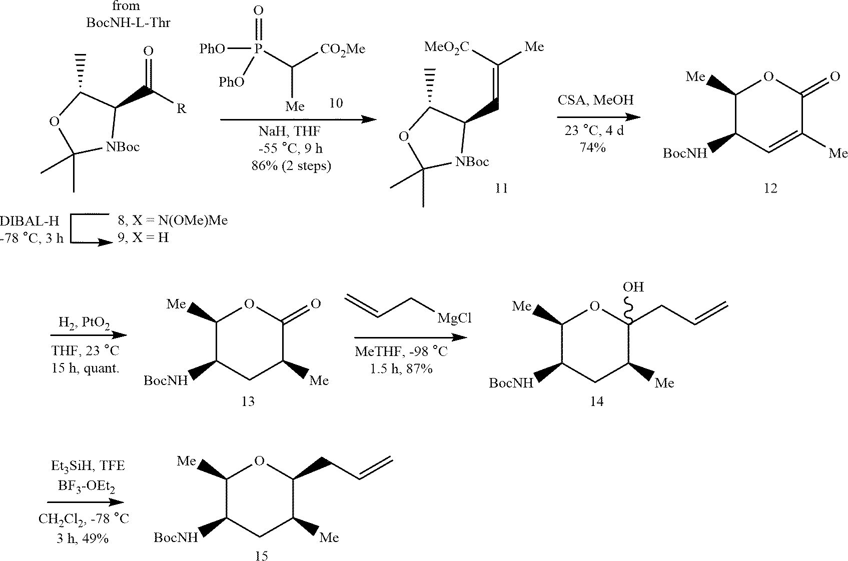

The simplest of the three subunits, left-hand subunit 22, was assembled by several approaches, two of which are illustrated in Scheme 3, with selection of the approach employed depending on the final target structure (e.g., meayamycin 1 vs analogues). For meayamycin (1) itself, we first implemented a straightforward approach starting with the commercially available optically active alkyne alcohol 16. Its acetal protection with ethyl vinyl ether (1.1 equiv, 0.1 equiv PPTS, CH₂Cl₂, 23° C., 2 h, 96%), alkyne carboxylation with Boc₂O and acetal deprotection without intermediate purification of 18 (80%, 2 steps), alcohol acetylation (82%) followed by stereoselective reduction of the alkyne to the cis alkene with Lindlar’s catalyst and subsequent t-butyl ester deprotection (96%, 2 steps) provided 22. Further, the conversion of 16 to 20 could be accomplished without purification of intermediates to provide 20 in yields as high as 81% and 22 in 78% overall yield (6 steps). A more versatile subunit 27, permitting late stage divergent (Ref. 22) functionalization, was also employed and was prepared with small variations on the approach described by in Ref. 5 and itself adopted from earlier unrelated studies. (Ref. 23). Commercially available optically active ethyl L-lactate protected as its TBDPS ether (Ref. 23d) (1.08 equiv TBDPSCI, 1.6 equiv imidazole, CH₂CI₂, 23° C., 2 h, 98%) was reduced with DIBAL-H (Et₂O, -78° C., 3 h) to the aldehyde 24 (Ref. 23d) and subjected to Z-selective Wadsworth-Horner-Emmons olefination with 25 (Ref. 21) (THF, -78 to -55° C., 10 h), affording 26 in superb yield (95%, two steps) and stereoselectivity (>99:1 Z:E). Hydrolysis of the methyl ester (5 equiv LiOH, MeOH/H₂O, 23° C., 24 h, 81%) completed the synthesis of 27, which was conducted on gram scales and required 4 steps (75% overall) from ethyl L-lactate.

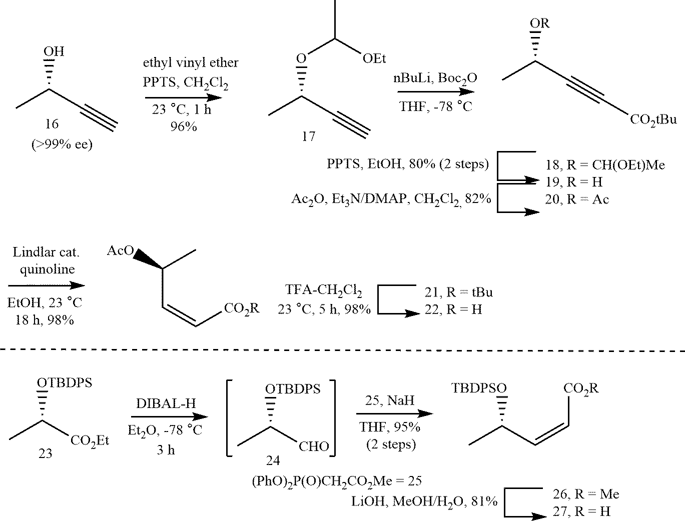

The initial assemblage of the subunits for elaboration to meayamycin paralleled the approach developed by Koide (Ref. 6) and is summarized in Scheme 4. Deprotection of 15 (10% TFA—CH₂Cl₂, 23° C.) followed by acylation of the liberated amine with 22 (1.2 equiv HATU, 4 equiv iPr₂NEt, MeCN, 23° C., 3-6 h, 70%) provided 28. Cross metathesis of 28 with methacrolein (20 equiv) provided the α,β-unsaturated aldehyde 29 (0.2 equiv Grubbs II catalyst (Ref. 24), CH₂Cl₂, 23° C., 36 h, 60% (36-60 h, 60-80%) or 0.01 equiv Grela catalyst (Ref. 25), CH₂Cl₂, 23° C., 12 h, 60%) followed by Wittig olefination afforded 30 (1.5 equiv Ph₃P⁺Me Br^{—}, 1.4 equiv tBuOK, THF, 0-23° C., 4 h, 57%). Final cross metathesis of 30 with the right-hand subunit 7 (0.2 equiv Grela catalyst, 0.3 equiv benzpquinone (p-BQ), ClCH₂CH₂Cl, 45° C., 12 h) notably without the protection of the secondary alcohol provided meayamycin (1) in yields as high as 52% provided. This completed a synthesis of 1 by an approach that required a longest linear sequence of 12 steps (22 steps overall) from commercially available materials and introduced what we suggest is the simplest, most straightforward preparation of a right-hand subunit for the class detailed to date.

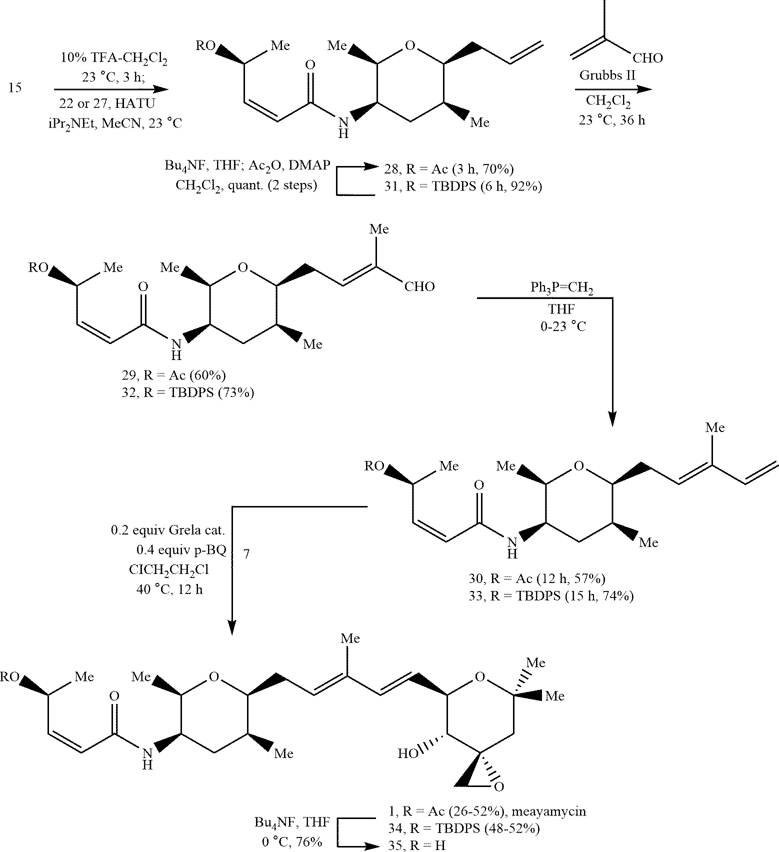

An alternative and further improved approach is also summarized in Scheme 4 and enlisted 27 in place of 22. Introduction of the meayamycin O-acetyl group may be accomplished at any stage in this alternative sequence and is exemplified with the conversion of 31 to 28 by silyl ether deprotection and acetylation. It uses the left-hand subunit 27 available in four simple steps, proceeds in higher overall yields because of the more robust stability of the alcohol substituent, avoids a problematic Z to E isomerization of the left-hand subunit that we observed during the coupling introduction of 22 to provide 28, and permits a late stage diversification of the terminal O-acyl substituent. Without optimization, this latter feature is exemplified by the preparation of carbamate 36 from 34 (Bu₄NF, THF, 0° C.; 1.5 equiv carbonyldimidazole (CDI), 0.2 equiv 4-dimethylaminopyridine (DMAP), CH₂CI₂, 23° C. then N-methylpiperazine; 42% overall), as shown below.

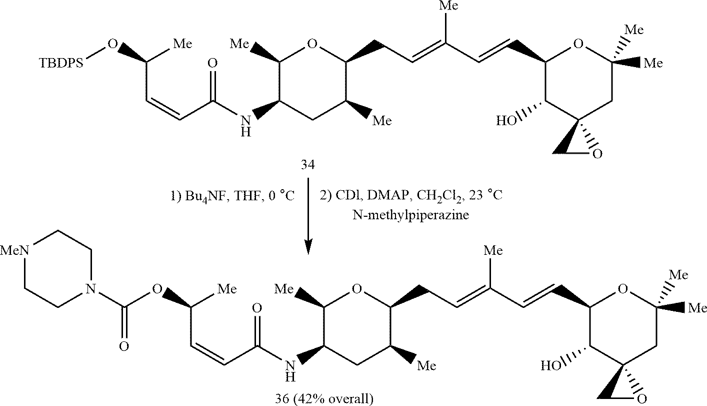

Referring to Scheme 5, a series of meayamycin O-acyl analogues was prepared from the alcohol derived from acetate hydrolysis of 30 (K₂CO₃, MeOH, 0° C., 95%) or later by TBDPS ether deprotection of 33 (3 equiv Bu₄NF, THF, 23° C., 4 h, quant.), its acylation to access alternative esters or the more stable carbamates (Ref. 12), and final cross metathesis of the resulting 37a-f with 7.

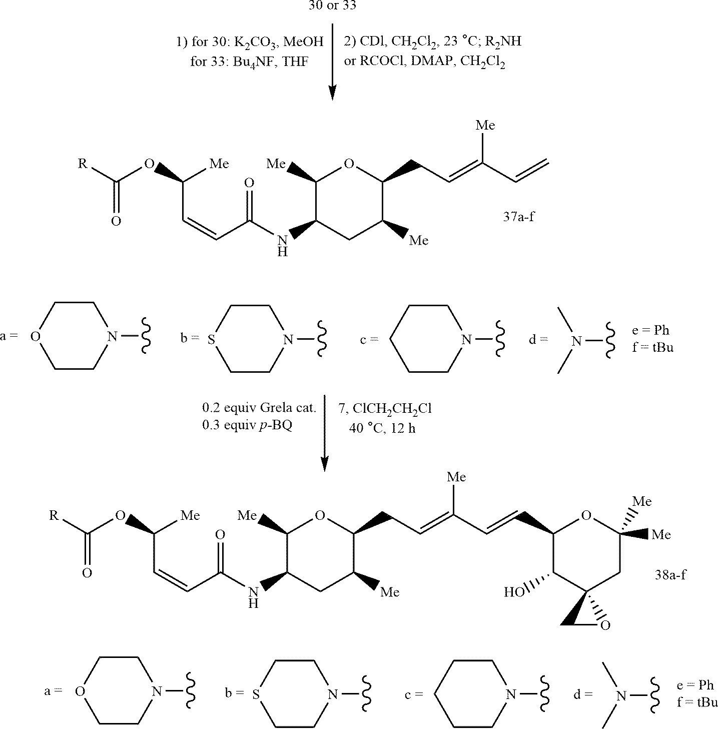

Additionally, we reexamined an alternative sequence for assembling 33 from the same three subunits (Scheme 6). This entailed first elaborating 15 to the diene 40 through cross metathesis with acrolein and subsequent Wittig olefination prior to introduction of the left-hand amide. In the absence of the labile acetate and sensitive Z-alkene found in the left-hand subunit, these transformations proved easier to implement and proceeded in higher conversions. In prior studies, an acid-catalyzed Boc deprotection (10% TFA, CH₂Cl₂, 23° C.) conducted on 40 was shown to result in competitive diene isomerization (Ref. 6) and we confirmed these observations. However, we found that Boc deprotection under alternative conditions (1 N HCl, EtOAc, 23° C., 15 min) did not suffer the same competitive isomerization and the liberated free amine could be coupled with 27 (HATU, iPr₂NEt, MeCN) to provide 33 in excellent yields, providing a viable and preferred alternative in future studies.

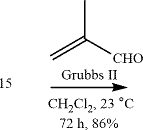

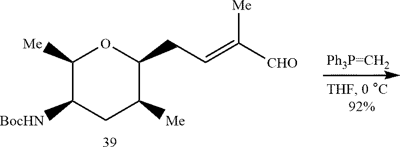

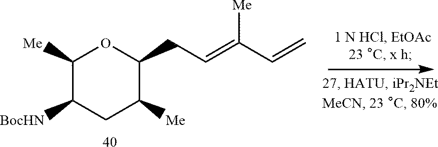

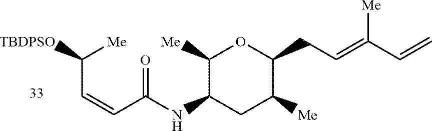

### Biological Activity

The results of the initial evaluation for cytotoxicity against various cancer cell lines of analogues 38a-f, along with those for 1, 36 and intermediate alcohol 35 are summarized in Table A. L1210 is a murine lymphocytic leukemia cell line. HCT116 and HCT116/VM46 are human colon cancer cell lines, with the latter being a multi-drug resistant variant of the former. MCF-7 is a human breast cancer cell line.

Whereas the removal of the O-acyl substituent substantially reduced potency (35) as previously disclosed in Ref. 12 and the incorporation of basic functionality with 36 led to a pronounced reduction in activity likely due to impeded cell penetration, all other replacements for the acetate itself (38a-f) largely matched the activity of 1, including the more stable carbamates. Notably, even the sterically large pivolate ester 38f displayed good albeit somewhat attenuated activity (5-fold less potent than 1), being more potent that the free alcohol 35 that lacks an acyl group. These observations complement those disclosed initially by Koide (Ref. 12) and Webb (Ref. 11) and more recently by Nicolaou (Ref. 10) on the thailanstatins and help define a site and functionality available for productive modification on the stand-alone drugs themselves or for linkage as antibody-drug conjugates.

Table A - Cytotoxicity of Compounds

| Compound | Cell Line (IC₅₀, nM) | | | |
|---|---|---|---|---|
| | L1210 | HCT116 | HCT116/ VM46 | MCT-7 |
| Meayamycin (1) | 0.54 | 0.56 | 0.51 | 0.31 |
| 35 (R = OH) | 7.8 | 9.7 | 7.4 | 47 |
| Meayamycin B (38a) | 0.51 | 0.49 | 0.55 | 0.39 |
| 38b | 0.60 | 0.48 | 0.52 | 0.42 |
| 38c | 0.44 | 0.68 | 0.59 | 2.7 |
| 38d | 0.58 | 0.54 | 0.53 | 0.39 |
| 38e | 1.8 | 0.72 | 0.65 | 2.4 |
| 38f | 1.2 | 3.5 | 2.4 | nd |
| 36 | 790 | 630 | 840 | 930 |

### EXAMPLES

The practice of this invention can be further understood by reference to the following examples, which are provided by way of illustration and not of limitation.

### Example 1 - Synthesis of the Right Hand Subunit

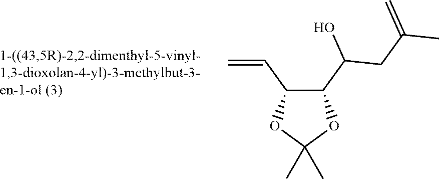

A solution of aldehyde 2 (Ref. 18) (368 mg, 2.36 mmol 1.0 equiv) in THF (40 mL) at 0° C. was treated with 2-methylallylmagnesium (9.4 mL of a 0.5 M solution, 4.72 mmol, 2.0 equiv) dropwise over 1 h. The mixture was allowed to warm to room temperature and stirred for 4 h before the reaction was quenched with the addition of saturated aqueous NH₄CI (10 mL) and extracted with EtOAc (3 x 20 mL). The combined organic phases were dried over Na₂SO₄, filtered, and concentrated in vacuo. The crude material was purified by column chromatography (SiO₂, 20% EtOAc in hexanes) to provide 3 (326 mg, 1.54 mmol, 65%) as a yellow oil as a mixture of diastereomers. Major diastereomer: ¹H NMR (600 MHz, CDCl₃) δ 6.05 (ddd, J = 17.3, 10.4, 7.0 Hz, 1H), 5.42 (dt, J = 17.2, 1.5 Hz, 1H), 5.32 - 5.23 (m, 1H), 4.90 (t, J = 1.7 Hz, 1H), 4.84 - 4.78 (m, 1H), 4.68 (t, J = 6.7 Hz, 1H), 3.97 (dd, J = 8.1, 6.3 Hz, 1H), 3.75 (tt, J = 8.1, 2.5 Hz, 1H), 2.53 - 2.49 (m, 1H), 2.32 - 2.00 (m, 1H), 1.76 (s, 3H), 1.49 (s, 3H), 1.37 (s, 3H); ¹³C NMR (150 MHz, CDCl₃) δ 142.3, 134.5, 118.1, 114.0, 108.8, 80.7, 79.0, 67.4, 42.6, 27.9, 25.5, 22.6. minor diastereomer: ¹H NMR (600 MHz, CDCl₃) δ 6.01 (ddd, J = 17.3, 10.4, 7.0 Hz, 1H), 5.36 (dt, J = 17.2, 1.5 Hz, 1H), 5.32 - 5.29 (m, 1H), 4.90 (t, J = 1.7 Hz, 1H), 4.84 - 4.78 (m, 1H), 4.57 (dd, J = 8.2, 6.7 Hz, 1H), 4.05 (dd, J = 6.7, 5.1 Hz, 1H), 3.84 - 3.68 (m, 1H), 2.51 (dt, J = 14.1, 1.8 Hz, 1H), 2.27 - 2.04 (m, 1H), 1.75 (s, 2H), 1.53 (s, 3H), 1.40 (s, 3H); ¹³C NMR (150 MHz, CDCl₃) δ 142.1, 134.3, 119.7, 113.5, 108.8, 80.3, 79.3, 67.8, 42.3, 27.6, 25.2, 22.6. IR (neat) vₘₐₓ3460, 2985, 2935, 1647, 1374, 1215, 1166, 1050, 875 cm⁻¹; HRMS-TOF-ESI (m/z) [M + H]⁺ calculated for C₁₂H₂₁O₃ 213.1485, found 213.1488.

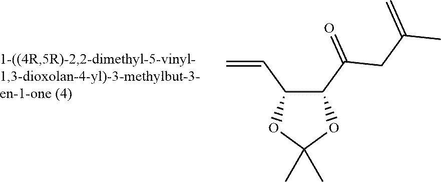

A solution of 3 (200 mg, 0.95 mmol, 1.0 equiv) in CH₂CI₂ (25 mL) at 0° C. was treated with DMP (605 mg, 1.43 mmol, 1.5 equiv) and the mixture was stirred for 3 h. After this time, the reaction was quenched with the addition of saturated aqueous NaHCO₃ (10 mL) and the mixture was extracted with CH₂Cl₂ (3 x 15 mL). The combined organic phase was washed with saturated aqueous NaCl, dried over Na₂SO₄, filtered, and concentrated in vacuo. The crude material was purified by column chromatography (SiO₂, 10% EtOAc in hexanes) to give 4 (176 mg, 0.84 mmol, 88%) as a yellow oil: ¹H NMR (600 MHz, CDCl₃) δ 5.68 (ddd, J = 17.0, 10.4, 6.5 Hz, 1H), 5.55 - 5.35 (m, 1H), 5.26 - 5.24 (m, 1H), 4.93 (s, 1H), 4.86 - 4.84 (m, 1H), 4.73 (s, 1H), 4.61 (d, J = 8.0 Hz, 1H), 3.32 (d, J = 17.2 Hz, 1H), 3.07 (d, J = 17.2 Hz, 1H), 1.73 (s, 3H), 1.64 (s, 3H), 1.40 (s, 3H); ¹³C NMR (150 MHz, CDCl₃) δ 207.1, 138.6, 132.4, 118.9, 115.3, 110.8, 83.1, 78.7, 49.4, 27.0, 25.0, 22.9; IR (neat) vₘₐₓ 2987, 1718, 1378, 1260, 1210, 1160, 1065, 872 cm⁻¹; HRMS-TOF-ESI (m/z) [M + Na]⁺ calculated for C₁₂H₁₈O₃Na 233.1154, found 233.1150.

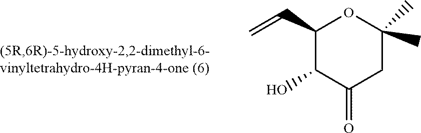

A solution of 4 (50 mg, 0.24 mmol, 1.0 equiv) in MeOH (0.3 mL) was treated with PPTS (12 mg, 0.048 mmol, 0.2 equiv) and the mixture was warmed at 60° C. until complete acetonide deprotection was observed by TLC. A solution of aqueous 1 N HCl (0.03 mL) was added and the mixture was stirred for an additional 2 to 4 h. The solvent was removed in vacuo to give the deprotected enone 5: ¹H NMR (600 MHz, CDCl₃) δ 6.23 - 5.98 (m, 1H), 5.72 (ddd, J = 17.2, 10.5, 5.8 Hz, 1H), 5.30 (dt, J = 17.1, 1.5 Hz, 1H), 5.22 (dt, J = 10.5, 1.4 Hz, 1H), 4.42 (ddt, J = 7.5, 3.0, 1.5 Hz, 1H), 4.36 (d, J = 3.6 Hz, 1H), 2.21 (d, J = 1.2 Hz, 3H), 1.98 (d, J = 1.3 Hz, 3H). The material was taken up in CHCl₃ (0.5 mL), Amberlyst-15 was added (10 mg) and the mixture was warmed at 80° C. for 12 h. The reaction mixture was filtered, and the solvent removed in vacuo. The crude material was purified by column chromatography (SiO₂, 5% EtOAc in hexanes) to give the 6 (38 mg, 0.18 mmol, 76%) as a clear oil. The spectroscopic data for 6 was consistent with that previously reported in the literature. (Ref. 12). For 6: ^{I}H NMR (500 MHz, CDCl₃) δ 6.05 (ddd, J = 17.3, 10.5, 5.3 Hz, 1H), 5.46 (dt, J = 17.1, 1.3 Hz, 1H), 5.35 (dt, J = 10.4, 1.2 Hz, 1H), 4.03 (s, 1H), 3.96 - 3.88 (m, 1H), 3.70 (s, 1H), 2.35 (s, 1H), 2.17 (s, 1H), 1.44 (s, 3H), 1.21 (s, 3H); HRMS-TOF-ESI (m/z) [M+H]⁺ calculated for C₉H₁₅O₃ 171.1021, found 171.1014.

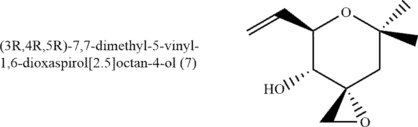

Compound 7 was synthesized following a literature procedure. (Ref. 19). The spectroscopic data for 7, obtained in 73% yield as a clear oil (157 mg) was consistent with that previously reported in the literature:

$${\left[α\right]}_{D}^{20} + 96 \left(c 1.0 , {\text{CHCl}}_{3}\right) ;$$

¹H NMR (600 MHz, CDCl₃) δ 5.98 (ddd, J = 17.0, 10.5, 6.2 Hz, 1H), 5.41 (dt, J = 17.3, 1.5 Hz, 1H), 5.29 (dt, J = 10.3, 1.3 Hz, 1H), 3.95 (ddt, J = 9.8, 6.2, 1.2 Hz, 1H), 3.51 (d, J = 8.8 Hz, 1H), 3.03 (d, J = 4.7 Hz, 1H), 2.49 (d, J = 4.7 Hz, 1H), 2.19 (dd, J = 14.3, 0.9 Hz, 1H), 1.63 (d, J = 10.4 Hz, 1H), 1.42 (d, J = 14.3 Hz, 1H), 1.40 (s, 3H), 1.28 (s, 3H); ¹³C NMR (150 MHz, CDCl₃) δ 136.7, 118.0, 74.8, 73.0, 68.0, 57.6, 47.7, 42.9, 31.1, 23.7. HRMS-TOF-ESI (m/z) [M+H]+ calculated for C₁₀H₁₇O₃ 185.1178, found 185.1171.

### Example 2 - Synthesis of the Central Subunit

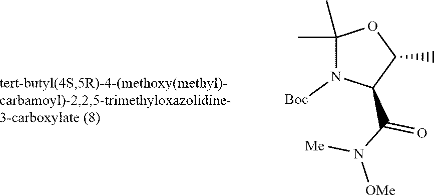

A solution of N-Boc-threonine (20.0 g, 0.091 mol, 1.0 equiv) in CH₂CI₂ (500 mL) was treated with iPr₂NEt (32 mL, 0.184 mol, 2.0 equiv), HOBT (14.8 g, 0.110 mol, 1.2 equiv) and EDCI (21.24 g, 0.111 mol, 1.22 equiv) at 0° C. The resulting solution was stirred at 23° C. for 22 h under Ar before being slowly quenched with addition of excess aqueous 1 M HCl at 0° C. The crude mixture was filtered over Celite and the organic layer separated. The aqueous layer was extracted with CH₂Cl₂ (3 x 150 mL) and the combined organic layer was washed with saturated aqueous NaHCO₃, saturated aqueous NaCl, dried over Na₂SO₄, filtrated and concentrated under reduced pressure provided the amide as a yellow oil.

A solution of the amide in THF (250 mL) was treated with PPTS (4.7 g, 0.019 mol, 0.2 equiv) and 2,2-dimethoxypropane (72 mL, 0.588 mol, 6.5 equiv) and the mixture was warmed at reflux for 18 h. After this time, the reaction was cooled and the solvent was removed under reduced pressure. The residue was taken up in EtOAc and H₂O and the aqueous layer was extracted three times with EtOAc. The combined organic layer was washed with saturated aqueous NaCl, dried over Na₂SO_{4,}, filtrated and concentrated in vacuo. The crude material was purified by column chromatography (SiO₂, 30% EtOAc in hexanes) to give 8 (23.4 g, 85%) as a colorless solid. All spectral data was consistent with reported data (Ref. 20). mp 34-35° C.;

$${\left[α\right]}_{D}^{20} - 10.2 \left(c 1.0 , {\text{CHCl}}_{3}\right) ;$$

¹H NMR (600 MHz, CDCl₃) δ 4.49 (d, J = 7.0 Hz, 0.4H), 4.37 (d, J = 7.0 Hz, 0.6H), 4.14 (dp, J = 25.1, 6.3 Hz, 1H), 3.78 (s, 1.5H), 3.72 (s, 1.6H), 3.21 (s, 3H), 1.65 (s, 1.7H), 1.61 (s, 1.6H), 1.59 (d, J = 3.4 Hz, 3H), 1.46 (s, 4H), 1.43 - 1.38 (m, 8H); ¹³C NMR (150 MHz, CDCl₃) δ 171.2, 170.5, 152.1, 151.3, 95.1, 94.6, 80.5, 80.3, 74.6, 74.3, 63.4, 63.3, 61.3 (2C), 61.2 (2C), 32.5, 28.6 (2C), 28.4, 27.0, 25.3, 24.2, 19.7, 19.5; IR (neat) vₘₐₓ 2976, 1696, 1678, 1363, 1170, 763, 614 cm⁻¹; HRMS-TOF-ESI (m/z) [M + H]⁺ calculated for C₁₄H₂₇N₂O₅ 303.1920, found 303.1921.

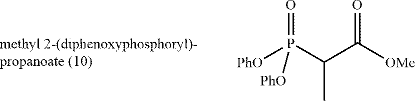

A solution of diphenyl phosphite (15 mL) in THF (10 mL) at 0° C. was treated with NaH (3.14 g of a 60% suspension in mineral oil, 0.079 mol, 1.0 equiv). After an hour at this temperature, methyl 2-bromoacetate (7.42 mL, 0.081 mmol, 1.02 equiv) in THF (20 mL) was added dropwise over 1 h and the mixture allowed to warm to room temperature and stirred for 15 h as 23° C. The reaction was quenched with the addition of saturated aqueous NH₄CI (16 mL) and the mixture was diluted with H₂O (20 mL) and Et₂O (40 mL). The layers were separated and the aqueous phase was extracted with EtOAc (3 x 30 mL). The combined organic phase was washed with saturated aqueous NaCl (40 mL), dried over Na₂SO₄, filtered and concentrated in vacuo.

The crude ester (8.49 g, 0.028 mol, 1.0 equiv) in DMSO (35 mL) and treated with NaH (1.12 g of a 60 % suspension in mineral oil, 0.028 mol, 1.0 equiv) and the mixture was stirred for 1 h. Methyl iodide (1.75 mL, 0.028 mol, 1.0 equiv) was added dropwise over 1 h and the reaction mixture was stirred for a further 2 h at 23° C. The reaction was quenched with the addition of saturated aqueous NH₄Cl (30 mL) and diluted with EtOAc (40 mL). The layers were separated, and the aqueous layer was extracted with EtOAc (2 x 30 mL). The combined organic layer was washed with saturated aqueous NaCl (35 mL), dried over Na₂SO₄, filtered and concentrated in vacuo. The crude material was purified by column chromatography (SiO₂, 10-20% EtOAc in hexanes) to give the 10 (4.17 g, 4.25 mmol, 98%) as a colorless oil identical to reported material (Ref. 21): ¹H NMR (600 MHz, CDCl₃) δ 57.34 - 7.28 (m, 4H), 7.21 - 7.15 (m, 6H), 3.75 (s, 3H), 3.38 (dq, J = 23.7, 7.3 Hz, 1H), 1.64 (dd, J = 19.2, 7.3 Hz, 3H); ¹³C NMR (150 MHz, CDCl₃) δ 169.3, 150.4, 150.3, 129.8, 125.4, 52.9, 40.0, 30.1, 11.9; IR (neat) vₘₐₓ2951, 1736, 1589, 1487, 1182, 1157, 759, 687 cm⁻¹; HRMS-TOF-ESI (m/z) [M + H]⁺ calculated for C₁₆H₁₈O₅P 321.0892, found 321.0894.

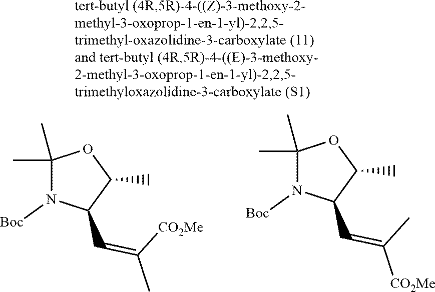

A solution of 8 (907 mg, 3.0 mmol, 1.0 equiv) in CH₂CI₂ was treated with DIBAL-H (5.02 mL of a 1.2 M solution in toluene, 6.0 mmol, 2.0 equiv) at -78° C. After 3 h at this temperature, the reaction was quenched with the addition of excess EtOAc. Saturated aqueous potassium sodium tartrate (20 mL) was added and the mixture was stirred for 3 h at 23° C. The layers were separated, and the aqueous phase was extracted with CH₂Cl₂ (2 x 15 mL). The combined organic phase was washed with saturated aqueous NaCl (20 mL), dried over Na₂SO₄, filtered and concentrated in vacuo. The crude aldehyde 9 was used without further purification.

A solution of 10 (1.16 g, 3.62 mmol, 1.2 equiv) in THF (5 mL) was treated with NaH (145 mg of a 60% suspension in mineral oil, 3.62 mmol, 1.2 equiv) at 0° C. and stirred for 1 h. After this time, the reaction mixture was cooled to -78° C. and a solution of crude 9 in THF (3 mL) was added dropwise over 30 min. The reaction mixture was warmed to -55° C. and stirred for a further 9 h before being quenched with the addition of saturated aqueous NH₄CI (4 mL). The organics were removed in vacuo and the residual aqueous phase was extracted with 10% EtOAc/hexanes. The combined organic phase was washed with saturated aqueous NaCl, dried over Na₂SO₄, filtered and concentrated in vacuo. The crude material was purified by column chromatography (SiO₂, 5-10% EtOAc in hexanes) to give the desired compound as a separable mixture of isomers, Z-isomer 11 (658 mg) and E-isomer S1 (143 mg) as colorless oils in 86% combined yield. For Z-11:

$${\left[α\right]}_{D}^{20} + 63 \left(c\right) \left(1.0 , {\text{CHCl}}_{3}\right) ;$$

¹H NMR (600 MHz, CDCl₃) δ 5.82 and 5.71 (br s, 1H), 4.93 and 4.79 (br s,1H), 3.90 -3.81 (m, 1H), 3.73 (br s, 3H), 1.95 (d, J = 1.5 Hz, 3H), 1.68 - 1.31 (m, 15H), 1.33 (d, J = 6.2 Hz, 3H); ¹³C NMR (150 MHz, CDCl₃) δ 167.5, 152.0, 143.2, 142.4, 128.5, 94.2, 93.6, 80.1, 79.5, 76.0, 75.4, 62.0, 61.7, 51.5, 36.7, 29.7, 28.3, 26.8, 26.6, 25.7, 24.7, 23.5, 20.6, 18.6, 18.1; IR (neat) vₘₐₓ2978, 1696, 1363, 1210, 1119, 1082, 859 cm⁻¹; HRMS-TOF-ESI (m/z) [M + H]⁺ calculated for C₁₆H₂₈NO₅ 314.1967, found 314.1963. For E-Sl:

$${\left[α\right]}_{D}^{20} + 19 \left(c 0.5 , {\text{CHCl}}_{3}\right) ;$$

¹H NMR (600 MHz, CDCl₃) δ 6.49 (br s, 1H), 4.18 and 4.08 (br s, 1H), 3.90 - 3.81 (m, 1H), 3.75 (br s, 3H), 1.93 and 1.89 (br s, 3H), 1.77 -1.29 (m, 15H), 1.26 (br d, J = 6.0 Hz, 3H); ¹³C NMR (150 MHz, CDCl₃) δ 168.1 (2C), 152.0, 151.8, 140.9, 140.0, 129.3, 128.7, 94.5, 94.0, 80.4, 79.9, 74.6, 62.2, 52.0, 36.7 (2C), 29.7, 28.3, 27.8, 26.3, 25.3, 17.5, 13.1, 12.8; IR (neat) vₘₐₓ 2977, 1697,1362, 1266, 1238, 1121, 937, 856 cm⁻¹; HRMS-TOF-ESI (m/z) [M + H]⁺ calculated for C₁₆H₂₈NO₅ 314.1967, found 314.1974.

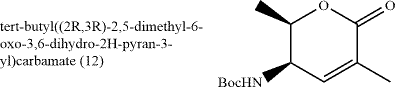

A solution of the E/Z mixture of 11 and S1 (15 g, 62.7 mmol, 1.0 equiv) in MeOH (480 mL) was treated with CSA (555 mg, 2.4 mmol, 0.04 equiv) and stirred for 4 days. After this time, the solvent was removed in vacuo and the residue was taken up in CH₂Cl₂ (250 mL), washed with saturated aqueous Na₂CO₃ (100 mL), saturated aqueous NaCl (50 mL), dried over Na₂SO₄, filtered and concentrated in vacuo. The crude material was purified by column chromatography (SiO₂, 10-30% EtOAc in hexanes) to give 12 (8.51 g, 74%) as a white solid: mp 149-150° C.;

$${\left[α\right]}_{D}^{20} - 176 \left(c \text{1} \text{.0,}\right) \left({\text{CHCl}}_{3}\right) ;$$

¹H NMR (600 MHz, CDCl₃) δ 6.61 (d, J = 6.3 Hz, 1H), 4.72 (d, J = 10.0 Hz, 1H), 4.59 (dq, J = 6.6, 2.9 Hz, 1H), 4.28 - 4.21 (m, 1H), 1.91 (s, 3H), 1.42 (s, 9H), 1.35 (d, J = 6.5 Hz, 3H); ¹³C NMR (150 MHz, CDCl₃) δ 165.5, 155.5, 138.2, 130.4, 80.4, 76.5, 46.3, 28.4, 17.1, 16.3; IR (neat) vₘₐₓ2990, 1704, 1507, 1158, 586 cm⁻¹; HRMS-TOF-ESI (m/z) [M + Na]⁺ calculated for C₁₂H₁₉NO₄Na 264.1212, found 264.1217.

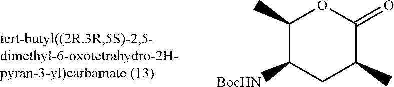

A solution of 12 (1.64 g, 6.8 mmol, 1.0 equiv) in THF (120 mL) was treated with PtO₂ (32.8 mg, 0.14 mmol, 0.02 equiv) and placed under an atmosphere of hydrogen. The reaction mixture was stirred for 15 h at 23° C. before being filtered through CELlTE™ and the solvent removed in vacuo to afford 13 (1.66 g, 6.8 mmol, 99%) as a white solid in 10:1 dr. For 13: mp 121-122° C.;

$${\left[α\right]}_{D}^{20} + 72 \left(c 1.0 , {\text{CHCl}}_{3}\right) ;$$

¹H NMR (600 MHz, CDCl₃) δ 4.75 (d, J = 9.4 Hz, 1H), 4.50 (dq, J = 6.4, 3.0 Hz, 1H), 4.15 - 4.04 (m, 1H), 2.68 - 2.52 (m, 2H), 1.43 (s, 9H), 1.34 (d, J = 6.4 Hz, 3H), 1.20 (d, J = 6.4 Hz, 3H); ¹³C NMR (150 MHz, CDCl₃) δ 175.7, 155.7, 80.0, 75.6, 48.0, 35.7, 32.5, 28.4, 16.2, 15.6; IR (neat) vₘₐₓ 2972, 1728, 1712, 1515, 1159, 592 cm⁻¹; HRMS-TOF-ESI (m/z) [M + Na]⁺ calculated for C₁₂H₂₁NOₐNa 266.1363, found 266.1360.

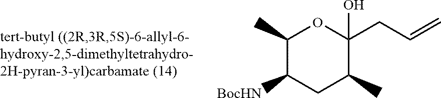

A solution of 13 (100 mg, 0.41 mmol, 1.0 equiv) in 2-methyltetrahydrofuran (2 mL) was treated with allylmagnesium chloride (0.4 mL of a 2 M solution in THF, 0.8 mmol, 1.95 equiv) at -98° C. and the mixture was stirred for 1.5 h before being quenched with the addition of saturated aqueous NH₄Cl (2 mL). The mixture was extracted with EtOAc (3 × 6 mL) and the combined organic phase was washed with saturated aqueous NaCl, dried over Na₂SO₄, filtered and concentrated in vacuo. The crude material was purified by column chromatography (SiO₂, 5-1.0% EtOAc in hexanes) to give 14 (116 mg, 0.35 mmol, 85% yield) as a colorless oil: ¹H NMR (600 MHz, CDCl₃) δ 5.93 (ddt, J = 17.1, 10.2, 6.9 Hz, 1H), 5.16 (d, J = 10.0 Hz, 1H), 5.13 (dq, J = 17.1, 1.6 Hz, 1H), 4.75 (br d, J = 9.8 Hz, 1H), 3.75 - 3.65 (m, 1H), 3.40 - 3.23 (m, 1H), 2.80 - 2.67 (m, 1H), 2.27 - 2.14 (m, 1H), 1.96 (ddd, J = 14.3, 9.9, 4.7 Hz, 1H), 1.43 (s, 9H), 1.14 (d, J = 6.3 Hz, 3H), 1.11 (d, J = 7.2 Hz, 3H); ¹³C NMR (150 MHz, CDCl₃) δ 212.8, 156.5, 130.9, 118.6, 79.2, 69.2, 53.8, 46.6, 42.5, 35.8, 28.4, 20.3, 18.0; IR (neat) vₘₐₓ2973, 1684, 1503, 1365, 1248, 1164, 1048, 917 cm⁻¹; HRMS-TOF-ESI (m/z) [M + Na]⁺ calculated for C₁₅H₂₇NO₄Na 308.1838, found 308.1838.

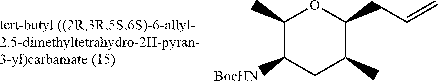

A solution of 14 (1.49 g, 5.22 mmol, 1.0 equiv) in CH₂Cl₂ (22.3 mL) was treated with 3,3,3-trifluoroethanol (TFE, 3.0 mL, 41.8 mmol, 8.0 equiv) and Et₃SiH (8.3 mL, 52.2 mmol, 10.0 equiv) at -78° C. After 30 min, BF₃ ▪ OEt₂ (2.58 mL, 20.9 mmol, 4.0 equiv) was added dropwise and the mixture was stirred for 4 h. After this time, the reaction was quenched with the addition of saturated aqueous NaHCO₃ (10 mL) and the mixture was extracted with CH₂CI₂ (3 x 15 mL). The combined organic phase was washed with saturated aqueous NaCl, dried over Na₂SO₄, filtered and concentrated in vacuo. The crude material was purified by column chromatography (SiO₂, 5-10% EtOAc in hexanes) to give 15 (687 mg, 2.56 mmol, 49%) as a colorless oil:

$${\left[α\right]}_{D}^{20} - 10.3 \left(c 1.2 , {\text{CHCl}}_{3}\right) ;$$

¹H NMR (600 MHz, CDCl₃) δ 5.77 (dddd, J = 16.6, 10.2, 7.7, 6.1 Hz, 1H), 5.09 (dq, J = 17.2, 1.7 Hz, 1H), 5.02 (br d, J = 10.2 Hz, 1H), 4.74 (br d, J = 9.6 Hz, 1H), 3.62 - 3.52 (m, 2H), 2.34 - 2.26 (m, 1H), 2.14 - 2.06 (m, 1H), 1.96 - 1.83 (m, 2H), 1.77 - 1.69 (m, 1H), 1.42 (s, 9H), 1.13 (d, J = 6.4 Hz, 3H), 1.01 (d, J = 7.4 Hz, 3H); ¹³C NMR (150 MHz, CDCl₃) δ 156.0, 135.0, 116.7, 80.7, 79.1, 76.5, 48.4, 37.6, 36.2, 29.0, 28.5, 17.8, 15.0; IR (neat) vₘₐₓ2976, 1714, 1492, 1227, 1055, 990, 913 cm⁻¹; HRMS-TOF-ESI (m/z) [M + H]⁺ calculated for C₁₅H₂₈NO₃ 270.2069, found 270.2071.

### Example 3 - Synthesis of Left-Hand Subunits

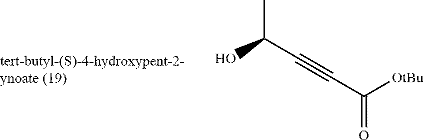

Ethyl vinyl ether (0.75 mL, 7.84 mmol, 1.1 equiv) was added to a solution of (S)-(-)-3-butyn-2-ol (500 mg, 7.13 mmol, 1.0 equiv) in CH₂Cl₂ (5.0 mL) followed by PPTS (180 mg, 0.713 mmol, 0.1 equiv). The reaction mixture was stirred for 2 h at 23° C. before being diluted with Et₂O (30 mL) and saturated aqueous NaCl (10 mL). The layers were separated, and the organic phase was dried over Na₂SO₄, filtered and concentrated in vacuo to give 17 as a mixture of diastereomers (972 mg, 6.83 mmol, 96%) as a clear oil. The spectroscopic data was consistent with literature values (Ref. 26). ¹H NMR (600 MHz, CDCl₃) δ 4.96 (q, J = 5.3 Hz, 0.5H), 4.85 (q, J = 5.3 Hz, 0.5H), 4.49 (qd, J = 6.7, 2.1 Hz, 0.5H), 4.34 (qd, J = 6.6, 2.1 Hz, 0.5H), 3.78 - 3.71 (m, 0.5H), 3.66 - 3.57 (m, 0.5H), 3.55 - 3.49 (m, 1H), 2.39 (d, J = 2.1 Hz, 0.5H), 2.39 (d, J = 2.1 Hz, 0.5H), 1.45 (dd, J = 6.6, 2.9 Hz, 3H), 1.34 (dd, J = 5.3, 3.0 Hz, 3H), 1.21 (td, J = 7.1, 0.9 Hz, 3H); HRMS-TOF-ESI (m/z) [M + Na]⁺ calculated for C₈H₁₄O₂Na 165.0891, found 165.0887.

A solution of 17 (970 mg, 6.8 mmol, 1.0 equiv) in THF (10 mL) at -78° C. was treated dropwise with n-butyllithium (2.9 mL of a 2.5 M solution in hexanes, 7.2 mmol, 1.05 equiv). After 15 min at this temperature, di-tert-butyl dicarbonate (1.64 mL, 7.2 mmol, 1.05 equiv) was added over 5 min and the mixture was stirred and allowed to warm to room temperature. The reaction mixture was diluted with Et₂O (15 mL) and washed with H₂O (10 mL) and saturated aqueous NaCl (10 mL). The organic phase was dried over Na₂SO₄, filtered and concentrated to give crude 18 as a dark oil. HRMS-TOF-ESI (m/z) [M + Na]⁺ calculated for C₁₃H₂₂O₄Na 265.1416, found 265.1415.

The ester 18 was immediately taken up in MeOH (12 mL), PPTS (170 mg, 0.68 mmol, 0.1 equiv) was added and the mixture was warmed at reflux for 2 h. After this time, the reaction mixture was cooled, diluted with Et₂O (15 mL), washed with saturated aqueous NaCl (5 mL), dried over Na₂SO₄, filtered and concentrated. The crude material was purified by column chromatography (SiO₂, 20% EtOAc in hexanes) to give 19 (926 mg, 5.44 mmol, 80% over 2-steps) as a clear oil:

$${\left[α\right]}_{D}^{20} - 22 \left(c 1.0 , {\text{CHCl}}_{3}\right) ;$$

¹H NMR (600 MHz, CDCl₃) δ 4.61 (q, J = 6.7 Hz, 1H), 1.97 (bs, 1H), 1.51 (d, J = 6.7 Hz, 3H), 1.49 (s, 9H) ); ¹³C NMR (150 MHz, CDCl₃) δ 152.6, 86.1, 83.9, 77.3, 58.2, 28.1, 23.5; IR (neat) vₘₐₓ 3386, 1705, 1369, 1257, 1155, 1067 cm⁻¹; HRMS-TOF-ESI (m/z) [M+H]⁺ calculated for C₉H₁₅O₃ 171.1021, found 171.1020.

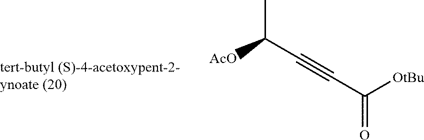

A stirred solution of 19 (900 mg, 5.29 mmol, 1.0 equiv) in CH₂CI₂ (18 mL) was treated with acetic anhydride (1.5 mL, 15.9 mmol, 3.0 equiv), Et₃N (3.7 mL, 26.5 mmol, 5 equiv) and DMAP (129 mg, 1.06 mmol, 0.2 equiv). After 12 h, saturated aqueous NH₄Cl was added and the mixture was extracted with EtOAc (3 x 25 mL). The combined organic phase was washed with saturated aqueous NaCl (20 mL), dried over Na₂SO₄, filtered and concentrated in vacuo. The crude material was purified by column chromatography (SiO₂, 10% EtOAc in hexanes) to give 20 (920 mg, 4.34 mmol, 82%) as a clear oil.

$${\left[α\right]}_{D}^{20} - 122 \left(c 1.0 , {\text{CHCl}}_{3}\right) ;$$

¹H NMR (600 MHz, CDCl₃) δ5.51 (q, J = 6.8 Hz, 1H), 2.09 (s, 3H), 1.53 (d, J = 6.8 Hz, 3H), 1.49 (s, 9H); ¹³C NMR (150 MHz, CDCl₃) δ 169.8, 152.2, 84.0, 82.5, 77.6, 59.6, 28.1, 21.0, 20.6; IR (neat) vₘₐₓ 1748, 1709, 1370, 1277, 1226, 1157, 1051 cm⁻ ¹; HRMS-TOF-ESI (m/z) [M + Na]⁺ calculated for C₁₁H₁₆O₄Na 235.0946, found 235.0942.

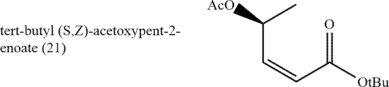

A solution of Lindlar’s catalyst (92 mg, 10% w/w) and quinoline (0.056 mL, 0.43 mmol, 0.1 equiv) was stirred in EtOH under an atmosphere of H₂ for 15 min before the addition of 20 (920 mg, 4.34 mmol, 1.0 equiv). After 16 h, the reaction mixture was filtered and concentrated in vacuo. The crude material was purified by column chromatography (SiO₂, 10% EtOAc in hexanes) to give 21 (911 mg, 4.25 mmol, 98%) as a pale-yellow oil:

$${\left[α\right]}_{D}^{20} + 4.8 \left(c 1.0 , {\text{CHCl}}_{3}\right) ;$$

¹H NMR (600 MHz, CDCl₃) δ6.26 - 6.21 (m, 1H), 6.02 (dd, J = 11.7, 7.7 Hz, 1H), 5.69 (dd, J = 11.7, 1.4 Hz, 1H), 2.04 (s, 3H), 1.49 (s, 9H), 1.36 (d, J = 6.5 Hz, 3H); ¹³C NMR (150 MHz, CDCl₃) δ 170.4, 164.9, 146.7, 121.9, 81.1, 68.7, 28.3, 21.4, 19.9; IR (neat) vₘₐₓ2978, 1741, 1712, 1368, 1235, 1158, 1117, 1048, 848. 822 cm⁻¹; HRMS-TOF-ESI (m/z) [M + Na]⁺ calculated for C₁₁H₁₈O₄Na 237.1103, found 237.1101.

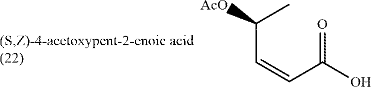

The ester 21 (210 mg, 0.98 mmol, 1.0 equiv) was stirred in TFA/CH₂Cl₂ (1.5 mL of a 10% solution) for 2 h before the solvent was removed in vacuo. The crude material was purified by column chromatography (SiO₂, 80% EtOAc in hexanes) to give 22 (152 mg, 0.96 mmol, 98%) as a clear oil:

$${\left[α\right]}_{D}^{20} + 21 \left(c 1.0 , {\text{CHCl}}_{3}\right) ;$$

¹H NMR (600 MHz, CDCl₃) δ10 26 (bs, 1H), 6.26 - 6.20 (m, 2H), 5.80 (d, J = 10.4 Hz, 1H), 2.05 (s, 3H), 1.37 (d, J = 6.3 Hz, 3H); ¹³C NMR (150 MHz, CDCl₃) δ 170.7, 170.6, 150.8, 119.3, 68.9, 21.3, 19.7; IR (neat) vₘₐₓ 2983, 1702, 1649, 1429, 1371, 1237, 1095, 1046, 892, 827 cm⁻¹; HRMS-TOF-ESI (m/z) [M + Na]⁺ calculated for C₇H₁₀O₄Na 181.0478, found 181.0478.

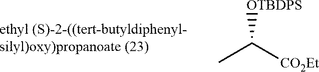

A solution of ethyl-L-lactate (2.0 g, 16.9 mmol) in CH₂Cl₂ (30 mL) was treated with imidazole (1.8 g, 26.4 mmol) and TBDPSCI (4.76 mL, 18.3 mmol, 1.08 equiv) at 0° C. The reaction mixture was stirred for 12 h at 23° C. before being quenched with the addition of H₂O. The organic layer was separated, and the aqueous layer was extracted three times with EtOAc. The combined organic layer was washed with saturated aqueous NaCl, dried over Na₂SO₄, filtered and concentrated under reduced pressure. Flash chromatography (SiO₂, 2-5% EtOAc in hexane) provided 23 (5.94 g, 98%) as colorless oil. All spectral data of synthetic 23 were identical with reported data:^{23c} [α]²⁶_{D} -45 (c 1.0, CH₂Cl₂); ¹H NMR (600 MHz, CDCl₃) δ 7.74 - 7.63 (m, 4H), 7.47 - 7.34 (m, 6H), 4.29 (q, J = 6.7 Hz, 1H), 4.04 (dq, J = 7.1, 2.4 Hz, 2H), 1.39 (d, J = 6.7 Hz, 3H), 1.16 (t, J = 7.1 Hz, 3H), 1.11 (s, 9H); ¹³C NMR (150 MHz, CDCl₃) 173.9, 136.0, 135.9, 133.8, 133.4, 129.9 (2C), 127.7 (2C), 69.1 (2C), 60.7, 27.0, 21.4, 19.4, 14.2; IR (neat) vₘₐₓ 2932, 2858, 1751, 1733, 1106, 699, 609 cm⁻¹.

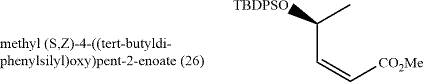

A solution of 23 (3.0 g, 8.41 mmol) in Et₂O (30 mL) was treated with 1 M DIBAL-H (12.6 mL, 12.6 mmol) at -78° C. The reaction mixture was stirred for 3 h at -78° C. before being quenched with the addtion of EtOAc. The resulting mixture was treated with excess saturated aqueous pottassium sodium tartrate, and stirred for 1 h at 23° C. After separation of organic layer, the aqueous layer was extracted three times with EtOAc. The combined organic layer was washed with saturated aqueous NaCl, dried over Na₂SO_{4,}, filtered and concentrated under reduced pressure. The obtained aldehyde 24 was used next step without purification.

A solution of methyl 2-(diphenoxyphosphoryl)acetate (25, 3.10 g, 10.1 mmol) in THF (20 mL) was treated with NaH (60% suspension in mineral oil, 390 mg, 9.75 mmol) at 0° C. and stirred for 1 h. The resulting mixture was treated with 24 in THF (10 mL) dropwised over 0.5 h at -78° C. and stirred for 9 h at -55° C. before being quenched with the addition of saturated aqueous NH₄Cl. After removal of THF under reduced pressure, EtOAc was added and the organic layer was separated. The aqueous layer was extracted three times with EtOAc. The combined organic layer was washed with saturated aqueous NaCl, dried over Na₂SO_{4,}, filtered and concentrated under reduced pressure. Flash chromatography (SiO₂, 2 % EtOAc/hexane) provided 32 (2.95 g, 95 %, > 99% Z) as colorless oil. All spectral data of synthetic 26 were identical with reported data (Ref. 23D): [α]²⁶_{D} +47 (c 1.0, CHCl₃); ¹H NMR (600 MHz, CDCl₃) δ 7.69 - 7.60 (m, 4H), 7.44 - 7.31 (m, 6H), 6.27 (dd, J = 11.7, 7.8 Hz, 1H), 5.52 (dd, J = 11.7, 1.4 Hz, 1H), 5.46 - 5.39 (m, 1H), 3.54 (s, 3H), 1.27 (d, J = 6.4 Hz, 3H), 1.08 (s, 9H); ¹³C NMR (150 MHz, CDCl₃) δ166.0, 154.1, 135.9 (2C), 134.3, 134.2, 129.7 (2C), 127.6 (2C), 116.6, 66.8, 51.2 (2C), 27.1, 23.4, 19.3; IR (neat) vₘₐₓ 2857, 1721, 1198, 1110, 1069, 699, 612 cm⁻¹.

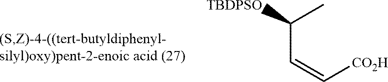

A solution of 26 (2.32 g, 6.29 mmol) in MeOH/H₂O (9:1, 46.4 mL) was treated with LiOH · H₂O (1.32 g, 31.5 mmol, 5 equiv) at 23° C. The reaction mixture was stirred for 24 h at 23° C. before being quenched with the addition of aqueous 0.5 M HCl. EtOAc was added to the quenched mixture, and the organic layer was separated (aqueous layer pH = 2). The aqueous layer was extracted three times with EtOAc. The combined organic layer was washed with saturated aqueous NaCl, dried over Na₂SO_{4,}, filtered and concentrated under reduced pressure. Flash chromatography (SiO₂, 20% EtOAc in hexane) provided 27 (2.00 g, 90%) as a colorless oil: [α]²⁶_{D} +40 (c 1.0, CHCl₃); ¹H NMR (600 MHz, CDCl₃) δ7.70 ― 7.60 (m, 4H), 7.41 - 7.30 (m, 6H), 6.37 (dd, J = 11.8, 7.8 Hz, 1H), 5.52 (dd, J = 11.8, 1.3 Hz, 1H), 5.41 ― 5.34 (m, 1H), 1.27 (d, J = 6.3 Hz, 3H), 1.09 (s, 9H); ¹³C NMR (150 MHz, CDCl₃) δ171.0, 156.2, 135.9, 134.9, 134.2, 133.9, 129.8, 127.9, 127.7, 127.6, 116.5, 66.7, 27.1, 23.3, 19.3; IR (neat) vₘₐₓ 2857, 1696, 1643, 1249, 1110, 1070, 698, 611 cm⁻¹; HRMS-TOF-ESI (m/z) [M-H]⁺ calculated for [C₂₁H₂₅O₃Si]⁺ 353.1573, found 353.1577.

### Example 4 ― Synthesis of Meayamycin

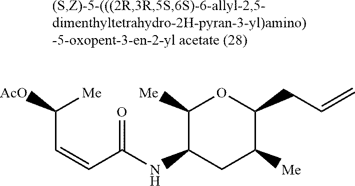

From 15. A solution of 15 (198 mg, 0.736 mmol) in CH₂Cl₂ (10 mL) was treated with trifluoroacetic acid (TFA, 1 mL) by syringe at 0° C. The reaction mixture was stirred for 3 h at 23° C. After completion of the deprotection reaction, the solvents were removed under reduced pressure. The residue was dissolved in CH₃CN (5 mL) and evaporated under reduced pressure. This procedure was repeated twice to completely remove TFA. A solution of 22 (140 mg, 0.886 mmol, 1.2 equiv) in CH₃CN (4.4 mL) was treated with iPr₂NEt (518 µL, 2.97 mmol) and O-(7-azabenzotriazol-1-yl)-N,N,N′,N′-tetramethyluronium hexafluorophoshate (HATU, 337 mg, 0.886 mmol, 1.2 equiv) at 0° C. The resulting mixture was stirred for 1 h at 0° C. before being added to the solution of the liberated free amine TFA salt in CH₃CN (6.6 mL). The reaction mixture was stirred for 15 h at 23° C., before being quenched with the addition of saturated aqueous NH₄Cl. After separation of the organic layer, the aqueous layer was extracted three times with EtOAc. The combined organic layer was washed with saturated aqueous NaHCO₃ and saturated aqueous NaCl, dried over Na₂SO₄, filtered and concentrated under reduced pressure. Flash chromatography (SiO₂, 10-30 % EtOAc in hexane) provided 28 (157 mg, 69%) as a colorless oil and the corresponding trans isomer (45 mg, 20%) as a colorless oil. All spectral data of synthetic 28 were identical with reported data (Ref. 6). [α]²¹_{D} -78 (c 0.5, CHCl₃); ¹H NMR (600 MHz, CDCl₃) δ6.25 - 6.17 (m, 1H), 6.00 (br d, J = 9.2 Hz, 1H), 5.83 (dd, J = 11.6, 7.9 Hz, 1H), 5.77 - 5.69 (m, 1H), 5.67 (d, J = 11.5 Hz, 1H), 5.04 (d, J = 17.1 Hz, 1H), 4.98 (d, J = 10.2 Hz, 1H), 3.93 - 3.87 (m, 1H), 3.60 (dq, J = 6.4, 2.2 Hz, 1H), 3.48 (dt, J = 7.1, 2.7 Hz, 1H), 2.32 - 2.22 (m, 1H), 2.10 - 2.03 (m, 1H), 1.99 (s, 3H), 1.92 - 1.83 (m, 2H), 1.76 ― 1.68 (m, 1H), 1.33 (d, J = 6.4 Hz, 3H), 1.09 (d, J = 6.5 Hz, 3H), 0.96 (d, J = 7.3 Hz, 3H); ¹³C NMR (150 MHz, CDCl₃) δ170.3, 164.8, 143.6, 134.7, 122.5, 116.7, 80.7, 75.9, 68.9, 47.1, 37.4, 35.9, 28.8, 21.2, 20.0, 17.8, 14.9; IR (neat) vₘₐₓ 2930, 1738, 1667, 1515, 1239, 1046, 813 cm⁻¹. HRMS-TOF-ESI (m/z) [M+H]⁺ calculated for [C₁₇H₂₈NO₄]⁺ 310.2018, found 310.2024.

For the trans isomer, (S,E)-5-(((2R,3R,55,65)-6-allyl-2,5-dimethyltetrahydro-2H-pyran-3-yl)amino)-5-oxopent-3-en-2-yl acetate: [α]²⁶_{D} -43 (c 1.0, CHCl₃); ¹H NMR (600 MHz, CDCl₃) δ6.69 (dd, J = 15.3, 5.3 Hz, 1H), 5.90 (dd, J = 15.4, 1.6 Hz, 1H), 5.82 (br d, J = 9.1 Hz, 1H), 5.76 - 5.68 (m, 1H), 5.48 - 5.40 (m, 1H), 5.06 (dd, J = 17.1, 1.7 Hz, 1H), 4.99 (d, J = 10.1 Hz, 1H), 3.97 - 3.90 (m, 1H), 3.61 (dq, J = 6.5, 2.2 Hz, 1H), 3.49 (dt, J = 7.1, 2.7 Hz, 1H), 2.32 - 2.23 (m, 1H), 2.12 - 2.03 (m, 1H), 2.02 (s, 3H), 1.89 (t, J = 3.6 Hz, 2H), 1.77 - 1.69 (m, 1H), 1.30 (d, J = 6.6 Hz, 3H), 1.08 (d, J = 6.5 Hz, 3H), 0.97 (d, J = 7.4 Hz, 3H); ¹³C NMR (150 MHz, CDCl₃) δ170.0, 164.7, 142.1, 134.7, 123.7, 116.8, 80.8, 76.0, 69.1, 47.3, 37.4, 35.9, 28.9, 21.2, 19.9, 17.9, 15.1; IR (neat) vₘₐₓ 2934, 1738, 1626, 1525, 1233, 1045, 631 cm⁻¹; HRMS-TOFESI (m/z) [M+H]⁺ calculated for [C₁₇H₂₈NO₄]⁺ 310.2018, found 310.2024.

From 31: A solution of 31 (187 mg, 0.37 mmol) in THF (2.6 mL) was treated with 1.0 M Bu₄NF in THF (1.1 mL, 1.1 mmol) at 0° C. and stirred for 4 h at 23° C. before being quenched with the addition H₂O. EtOAc was added and the organic layer was separated. The aqueous layer was extracted three times with EtOAc. The combined organic layer was washed with saturated aqueous NaCl, dried over Na₂SO₄, filtered and concentrated under reduced pressure. The TBDPS byproduct was removed by short flash chromatography (SiO₂, 20% EtOAc/hexane to EtOAc).

A solution of the pre-purified alcohol intermediate in CH₂Cl₂ (3.0 mL) was treated with Ac₂O (52.3 µL, 0.55 mmol), Et₃N (103 µL, 0.738 mmol) and DMAP (4.5 mg, 0.04 mmol) at 0° C. and stirred for 12 h at 23° C. before being quenched with the addition of saturated aqueous NH₄Cl. After separation of organic layer, the aqueous layer was extracted three times with EtOAc. The combined organic layer was washed with saturated aqueous NaHCO₃, saturated aqueous NaCl, dried over Na₂SO₄, filtrated and concentrated under reduced pressure. Flash chromatography (SiO₂, 20-30 % EtOAc in hexane) provided 28 (115.3 mg, quant.) as a colorless oil.

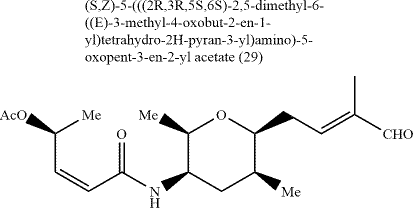

A solution of 28 (500 mg, 1.62 mmol) in CH₂Cl₂ (7.3 mL) was treated with methacrolein (2.68 mL, 32.4 mmol, 20 equiv) and Grubbs 2nd generation catalyst²⁴ (274 mg, 0.324 mmol, 0.2 equiv) at 23° C. The reaction mixture was stirred for 36 h at 23° C. The excess methacrolein and CH₂Cl₂ were removed under reduced pressure. Flash chromatography (SiO₂, 10-40 % EtOAc in hexane) provided 29 (338 mg, 60%) as brown oil. All spectral data of synthetic 29 were identical with reported data (Ref. 6): [α]²⁴_{D} -69 (c 0.5, CHCl₃); ¹H NMR (600 MHz, CDCl₃) δ9.42 (s, 1H), 6.56 ― 6.51 (m, 1H), 6.26 - 6.19 (m, 1H), 6.04 (br d, J = 9.0 Hz, 2H), 5.88 (dd, J = 11.6, 8.0 Hz, 1H), 5.73 (dd, J = 11.7, 1.3 Hz, 1H), 3.99 - 3.93 (m, 1H), 3.69 (dq, J = 6.5, 2.3 Hz, 1H), 3.68 - 3.63 (m, 1H), 2.60 ― 2.52 (m, 1H), 2.44 - 2.37 (m, 1H), 2.04 (s, 3H), 2.01-1.93 (m, 2H), 1,86 ― 1.78 (m, 1H), 1.76 (s, 3H), 1.38 (d, J = 6.5 Hz, 3H), 1.16 (d, J = 6.5 Hz, 3H), 1.06 (d, J = 7.4 Hz, 3H); ¹³C NMR (150 MHz, CDCl₃) δ195.0, 170.3, 164.9, 150.6, 143.3, 140.4, 122.5, 79.7, 76.0, 68.8, 46.8, 35.7, 32.7, 29.4, 21.2, 19.9, 17.7, 15.0, 9.4; IR (neat) vₘₐₓ 2934, 1731, 1668, 1639, 1242, 1047, 909, 729 cm⁻¹; HRMS (ESI+) calculated for C₁₉H₂₉NO₅ (M⁺) 351.2046, found 351.2041.⁶

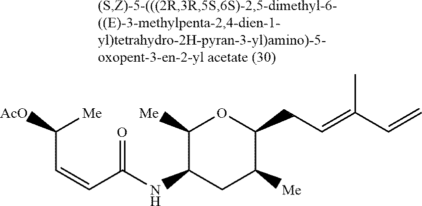

Methyltriphenylphosphonium bromide (687 mg, 1.92 mmol) in THF (1.3 mL) was treated with 1 M KO^{t}Bu in THF (1.73 mL, 1.73 mmol) at 0° C. and the solution was stirred for 1 h at 0° C. A solution of 29 (338.1 mg, 0.962 mmol) in THF (3 mL) was add dropwise to the Wittig reagent solution at 0° C. The reaction mixture was stirred for 12 h at 23° C. before being quenched with the addition of saturated aqueous NH₄Cl. After separation of the organic layer, the aqueous layer was extracted three times with EtOAc. The combined organic layer was washed with saturated aqueous NaCl, dried over Na₂SO₄, filtered and concentrated under reduced pressure. Flash chromatography (SiO₂, 10-40% EtOAc in hexane) provided 30 (190 mg, 57%) as colorless oil and recovered 29 (60.9 mg, 18%). All spectral data of synthetic 29 were identical with reported data (Ref. 6): [α]²⁶_{D} -74 (c 1.0, CHCl₃); ¹H NMR (600 MHz, CDCl₃) δ6.35 (dd, J = 17.3, 10.8 Hz, 1H), 6.29 - 6.21 (m, 1H), 6.02 (br d, J = 9.2 Hz, 1H), 5.88 (dd, J = 11.6, 7.9 Hz, 1H), 5.70 (dd, J = 11.6, 1.3 Hz, 1H), 5.45 (t, J = 7.2 Hz, 1H), 5.10 (d, J = 17.3 Hz, 1H), 4.94 (d, J = 10.6 Hz, 1H), 3.97 - 3.90 (m, 1H), 3.66 (dq, J = 6.5, 2.3 Hz, 1H), 3.53 (dt, J = 7.2, 2.8 Hz, 1H), 2.42 - 2.34 (m, 1H), 2.28 - 2.20 (m, 1H), 2.03 (s, 3H), 1.97 - 1.89 (m, 2H), 1.82 - 1.75 (m, 1H), 1.76 (s, 3H), 1.38 (d, J = 6.5 Hz, 3H), 1.14 (d, J = 6.5 Hz, 3H), 1.01 (d, J = 7.4 Hz, 3H); ¹³C NMR (150 MHz, CDCl₃) δ 170.4, 164.9, 143.6, 141.3, 135.7, 128.2, 122.6, 111.1, 80.9, 76.0, 68.9, 47.1, 35.9, 31.9, 28.9, 21.3, 20.0, 17.9, 15.1, 12.0; IR (neat) vₘₐₓ 2931, 1736, 1666, 1632,1046, 814 cm⁻¹; LRMS (m/z) [M+H]⁺ 350.3.

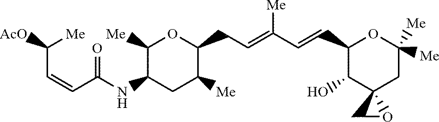

A solution of 30 (36 mg, 0.1 mmol, 1.0 equiv) in ClCH₂CH₂Cl (0.2 mL) at 45° C. was treated with p-benzoquinone (3.2 mg, 0.03 mmol, 0.3 equiv), Grela catalyst²⁵ (0.2 mL of 14 mg, 0.02 mmol, 0.2 equiv in 0.6 mL of ClCH₂CH₂Cl) and 7 (0.2 mL of 28 mg, 0.152 mmol, 1.5 equiv in 0.6 mL of ClCH₂CH₂Cl). The mixture was stirred for 1 h before a second portion of the solution of catalyst (0.2 mL) and 7 (0.2 mL) were added. After an additional 1 h, the final portion of catalyst (0.2 mL) and 7 (0.2 mL) were added. After 12 h, the reaction mixture was cooled and concentrated in vacuo. The crude material was purified by CombiFlash column chromatography (SiO₂, 20-50% EtOAc in hexanes gradient) to afford 1 (18.8 mg, 0.0372 mmol, 37%; typically 26-52%). For subsequent biological studies, semi-preparative reverse-phase HPLC purification of 1 was performed using a Nacalai Tesque, Inc., COSMOSIL, 5C18-AR-II column, 10 x 250 mm, 4.3 mL/min, 50% acetonitrile/water with 0.07% TFA to 100% acetonitrile, linear gradient from 0.5 min to 22 min, 100% acetonitrile for 3 min, retention time = 8.6 min. For 1: ¹H NMR (400 MHz, CD₂Cl₂) δ6.35 (dd, J = 15.8, 1.0 Hz, 1H), 6.31 ― 6.23 (m, 1H), 5.99 (m, 1H), 5.90 (dd, J = 11.6, 7.8 Hz, 1H), 5.71 (dd, J = 11.6, 1.3 Hz, 1H), 5.65 (dd, J = 15.7, 6.6 Hz, 1H), 5.57 - 5.49 (m, 1H), 4.01 ― 3.95 (m, 1H), 3.90 (ddq, J = 7.5, 3.0, 1.4 Hz, 1H), 3.66 (qd, J = 6.5, 2.5 Hz, 1H), 3.56 - 3.52 (m, 1H), 3.52 - 3.44 (m, 1H), 2.96 (d, J = 4.7 Hz, 1H), 2.46 (d, J = 4.7 Hz, 1H), 2.36 (dt, J = 14.5, 7.1 Hz, 1H), 2.28 - 2.20 (m, 1H), 2.16 (ddt, J = 14.3, 1.9, 0.9 Hz, 1H), 2.01 (s, 3H), 1.95 - 1.91 (m, 2H), 1.80 (t, J = 2.1 Hz, 1H), 1.78 (br s, 3H), 1.64 (d, J = 10.7 Hz, 1H), 1.40 (d, J = 14.3 Hz, 1H), 1.36 (s, 3H), 1.34 (d, J = 6.5 Hz, 3H), 1.24 (s, 3H), 1.11 (d, J = 6.4 Hz, 3H), 1.01 (d, J = 7.3 Hz, 3H); HRMS-TOF-ESI (m/z) [M+H]⁺calculated for C₂₈H₄₄NO₇ 506.3118, found 506.3118.

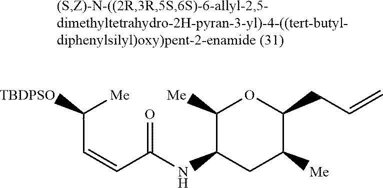

A solution of 15 (525 mg, 1.95 mmol) in CH₂Cl₂ (26 mL) was treated with trifluoroacetic acid (TFA, 2.6 mL) by syringe at 0° C. The reaction mixture was stirred for 3 h at 23° C. After completion of the deprotection reaction, the solvents were removed under reduced pressure. The residue was dissolved in CH₃CN (13 mL) and evaporated under reduced pressure. This procedure was repeated twice to completely remove TFA. A solution of 27 (829 mg, 2.34 mmol) in CH₃CN (10.6 mL) was treated with iPr₂NEt (1.37 mL, 7.86 mmol) and O-(7-azabenzotriazol-1-yl)-N,N,N′,N′-tetramethyluronium hexafluorophoshate (890 mg, 2.34 mmol) at 0° C. The resulting mixture was stirred for 1 h at 0° C. This solution was added dropwise to a solution of the TFA salt of the deprotected amine in CH₃CN (15 mL) at 0° C. The reaction mixture was stirred for 12 h at 23° C., before being quenched with the addition of saturated aqueous NH₄Cl. After separation of organic layer, the aqueous layer was extracted three times with EtOAc. The combined organic layer was washed with saturated aqueous NaHCO₃ and saturated aqueous NaCl, dried over Na₂SO₄, filtered and concentrated under reduced pressure. Flash chromatography (SiO₂, 10% EtOAc in hexane) provided 31 (906 mg, 92%) as white amorphous powder: [α]²⁶_{D} -37 (c 1.0, CHCl₃); ¹H NMR (600 MHz, CDCl₃) δ7.68 ― 7.63 (m, 4H), 7.41 - 7.29 (m, 6H), 6.10 (dd, J = 11.6, 7.4 Hz, 1H), 5.83 - 5.73 (m, 1H), 5.69 - 5.62 (m, 1H), 5.52 (d, J = 9.2 Hz, 1H), 5.40 (dd, J = 11.6, 1.4 Hz, 1H), 5.12 (dq, J = 17.1, 1.6 Hz, 1H), 5.05 (d, J = 10.2 Hz, 1H), 3.84 - 3.78 (m, 1H), 3.60 (dq, J = 6.5, 2.2 Hz, 1H), 3.51 (dt, J = 7.2, 2.8 Hz, 1H), 2.36 - 2.28 (m, 1H), 2.16 - 2.08 (m, 1H), 1.92 ― 1.84 (m, 1H), 1.84 - 1.71 (m, 1H), 1.28 (d, J = 6.3 Hz, 3H), 1.07 (t, J = 6.5 Hz, 3H), 1.06 (s, 9H), 0.95 (d, J = 7.4 Hz, 3H); ¹³C NMR (150 MHz, CDCl₃) δ164.9, 151.0, 135.9 (2C), 134.8, 134.7, 134.6, 129.6, 129.5, 127.6, 127.5, 119.1, 116.9, 80.8, 76.1, 67.1, 47.0, 37.5, 36.0, 29.0, 27.2, 23.9, 19.4, 17.9, 15.2; IR (neat) vₘₐₓ 2855, 1665, 1623, 1066, 822, 699, 612 cm⁻¹; HRMS-TOF-ESI (m/z) [M+H]⁺ calculated for [C₃₁H₄₄NO₃Si]⁺ 506.3090, found 506.3090.

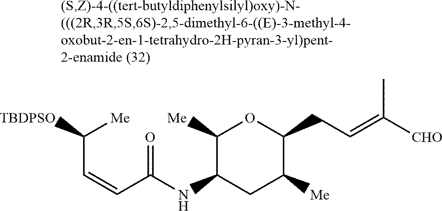

A solution of 31 (900 mg, 1.78 mmol) in CH₂Cl₂ (8 mL) was treated with methacrolein (2.95 mL, 35.6 mmol) and Grubbs 2nd generation catalyst (302 mg, 0.36 mmol, 0.2 equiv) at 23° C. The reaction mixture was stirred for 48 h at 23° C. The excess methacrolein and CH₂Cl₂ were removed under reduced pressure. Flash chromatography (SiO₂, 10-20% EtOAc in hexane) provided 32 (714 mg, 73%) as a colorless amorphous powder: [α]²⁶_{D} -40 (c 0.5, CHCl₃); ¹H NMR (600 MHz, CDCl₃) δ9.42 (s, 1H), 7.70 ― 7.59 (m, 4H), 7.41 - 7.28 (m, 6H), 6.52 (t, J = 6.6 Hz, 1H), 6.11 (dd, J = 11.6, 7.4 Hz, 1H), 5.70 - 5.61 (m, 1H), 5.53 (d, J = 9.2 Hz, 1H), 5.44 (d, J = 11.6 Hz, 1H), 3.66 - 3.58 (m, 2H), 2.59 - 2.49 (m, 1H), 2.44 - 2.35 (m, 1H), 1.94 - 1.82 (m, 2H), 1.82 - 1.77 (m, 1H), 1.78 (s, 3H), 1.29 (d, J = 6.2 Hz, 3H), 1.10 ― 1.04 (m, 12H), 0.98 (d, J = 7.4 Hz, 3H); ¹³C NMR (150 MHz, CDCl₃) δ195.1 (2C), 164.9, 151.0, 150.4, 140.6, 135.9, 135.8, 134.6, 134.5 129.5 (2C), 127.5 (2C), 119.0, 79.8 (2C), 76.3 (2C), 67.0, 46.7, 35.8, 32.8, 29.6, 27.1, 23.8, 19.3, 17.8, 15.3, 9.6; IR (neat) vₘₐₓ 2855, 1667, 1633, 1504, 1110, 1063, 997, 820, 610 cm⁻¹; HRMS-TOF-ESI (m/z) [M+H]⁺ calculated for [C₃₃H₄₆NO₄Si]⁺ 548.3196, found 548.3198.

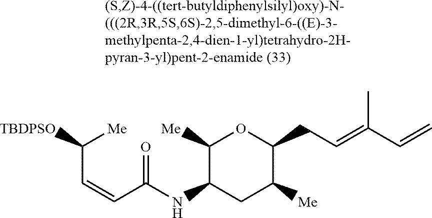

From 32: Methyltriphenylphosphonium bromide (932 mg, 2.61 mmol) in THF was treated with 1 M KO^{t}Bu in THF (2.35 mL, 2.35 mmol) at 0° C. and the mixture was stirred for 1 h at 0° C. A solution of 32 (714 mg, 1.30 mmol) in THF (7 mL) was added dropwise at 0° C. The reaction mixture was stirred for 15 h at 23° C. before being quenched with the addition of saturated aqueous NH₄Cl. After separation of organic layer, the aqueous layer was extracted three times with EtOAc. The combined organic layer was washed with saturated aqueous NaCl, dried over Na₂SO₄, filtered and concentrated under reduced pressure. Flash chromatography (SiO₂, 5-15 % EtOAc in hexane) provided 33 (528 mg, 74%) as a colorless wax: [α]²⁶_{D} -33 (c 0.5, CHCl₃); ¹H NMR (600 MHz, CDCl₃) δ7.70 ― 7.62 (m, 4H), 7.44 ― 7.28 (m, 6H), 6.38 (dd, J = 17.4, 10.7 Hz, 1H), 6.11 (dd, J = 11.6, 7.4 Hz, 1H), 5.71 ― 5.63 (m, 1H), 5.54 (d, J = 9.2 Hz, 1H), 5.46 (t, J = 7.2 Hz, 1H), 5.42 (d, J = 11.6 Hz, 1H), 5.12 (d, J = 17.4 Hz, 1H), 4.97 (d, J = 10.7 Hz, 1H), 3.86 - 3.78 (m, 1H), 3.61 (dq, J = 6.5, 2.2 Hz, 1H), 3.51 (dt, J = 7.3, 2.8 Hz, 1H), 2.44 - 2.34 (m, 1H), 2.29 - 2.20 (m, 1H), 1.92 ― 1.78 (m, 3H), 1.77 (s, 3H), 1.29 (d, J = 6.3 Hz, 3H), 1.10 ― 1.05 (m, 12H), 0.96 (d, J = 7.3 Hz, 3H); ¹³C NMR (150 MHz, CDCl₃) δ164.9, 150.9, 141.4 (2C), 135.9, 135.8 (2C), 134.6 (2C), 129.5 (2C), 128.2, 127.6, 127.5, 119.1, 111.3, 80.9, 76.1 (2C), 67.0, 47.0, 36.0, 32.0, 29.0, 27.1, 23.9, 19.4, 17.9, 15.3, 12.1(2C); IR (neat) vₘₐₓ 2929, 2855, 1663, 1628, 1501, 1110, 1063, 699, 610 cm⁻¹; HRMS-TOF-ESI (m/z) [M+H]⁺ calculated for [C₃₄H₄₈NO₃Si]⁺ 546.3403, found 546.3406.

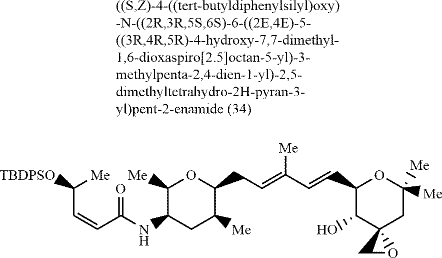

A solution of 33 (50 mg, 0.092 mmol, 1.0 equiv) in ClCH₂CH₂Cl (0.2 mL) at 40° C. was treated with p-benzoquinone (4 mg, 0.037 mmol, 0.4 equiv), Grela catalyst²⁵ (0.2 mL of 24 mg, 0.028 mmol, 0.3 equiv in 0.6 mL of ClCH₂CH₂Cl) and 7 (0.2 mL of 25 mg, 0.138 mmol, 1.5 equiv in 0.6 mL of ClCH₂CH₂Cl). The mixture was stirred for 1 h before a second portion of the solution of the catalyst (0.2 mL) and 7 (0.2 mL) were added. After an additional 1 h, the final portion of the catalyst (0.2 mL) and 7 (0.2 mL) were added. After 12 h, the reaction mixture was cooled and concentrated in vacuo. The crude material was purified by column chromatography over silica gel to give 34 (28.4 mg, 0.041 mmol, 44%): ¹H NMR (600 MHz, CDCl₃) δ7.70 ― 7.60 (m, 4H), 7.41 - 7.28 (m, 6H), 6.39 (d, J = 15.7 Hz, 0.5H), 6.09 (ddd, J = 13.4, 7.2, 3.5 Hz, 1H), 5.71 ― 5.59 (m, 1.5H), 5.50 (tt, J = 14.6, 8.3 Hz, 1H), 5.41 (ddd, J = 11.6, 4.9, 1.5 Hz, 1H), 4.02 (dd, J = 9.6, 6.7 Hz, 0.5H), 3.79 (ddt, J = 9.4, 4.7, 2.5 Hz, 1.5H), 3.63 ― 3.57 (m, 1H), 3.54 (t, J = 9.9 Hz, 1H), 3.48 (td, J = 7.3, 2.8 Hz, 1H), 3.03 (p, J = 5.1 Hz, 1H), 2.50 (t, J = 5.0 Hz, 1H), 2.37 (dq, J = 14.3, 7.7, 6.8 Hz, 1H), 2.25 - 2.16 (m, 1H), 1.85 (td, J = 10.1, 4.8 Hz, 1H), 1.80 (t, J = 2.3 Hz, 1H), 1.78 (s, 3H), 1.77 ― 1.71 (m, 2H), 1.62 (d, J = 10.1 Hz, 1H), 1.47 ― 1.42 (m, 3H), 1.41 (d, J = 5.3 Hz, 3H), 1.28 (d, J = 4.2 Hz, 3H), 1.26 (m, 3H), 1.05 (s, 9H), 0.94 (dd, J = 10.9, 7.3 Hz, 3H); HRMS-TOF-ESI (m/z) [M+H]⁺ calculated for C₄₂H₆₀NO₆Si 702.4184, found 702.4190.

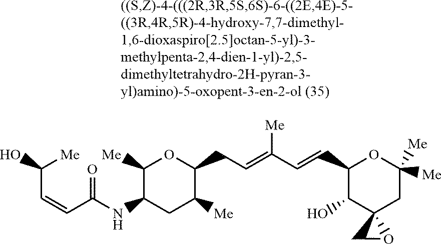

From 34: Tetra-n-butylammonium fluoride (Bu₄NF, 1.0 M in THF, 0.017 mL, 0.017 mmol, 1.2 equiv) was added to a solution of 34 (10 mg, 0.014 mmol, 1.0 equiv) in THF (0.5 mL) at 0° C. After stirring for 2 h, the reaction mixture was concentrated in vacuo. The crude material was purified by PTLC (100% EtOAc) to give 35 (7.6 mg, 0.0091 mmol, 65%): ¹H NMR (400 MHz, CD₂Cl₂) δ 6.44 - 6.32 (m, 1H), 6.26 - 6.06 (m, 2H), 5.88 (dd, J = 3.4, 1.7 Hz, 1H), 5.81 - 5.72 (m, 1H), 5.68 - 5.59 (m, 1H), 5.56 - 5.46 (m, 1H), 4.16-4.06 (m, 1H), 3.98 (dd, J = 3.4, 1.7 Hz, 1H), 3.95 (dd, J = 3.5, 1.8 Hz, 1H), 3.67 (m, 1H), 3.55 (m, 1H), 3.46 (m, 1H), 2.96 (d, J = 4.7 Hz, 1H), 2.46 (dd, J = 4.7, 2.0 Hz, 1H), 2.33 (s, 1H), 2.19 (d, J = 4.2 Hz, 1H), 2.15 (d, J = 4.2 Hz, 1H), 1.93 (m, 2H), 1.81 ― 1.75 (m, 4H), 1.44 -1.35 (m, 4H), 1.28 - 1.26 (m, 3H), 1.24 (m, 3H), 1.11 (dd, J = 6.5, 2.1 Hz, 3H), 1.05 - 1.02 (m, 3H); HRMS-TOF-ESI (m/z) [M+Na]⁺ calculated for C₂₆H₄₁NO₆Na 486.2826, found 486.2816.

From 1: Compound 1 was dissolved in a mixture of THF-CH₃OH-H₂O. LiOH-H₂O was added, and the reaction mixture was stirred at 23° C. The reaction mixture was then acidified to pH 1 with aqueous 1 N HCl, after which it was diluted with EtOAc. The organic layer was separated, washed with H₂O and saturated aqueous NaCl, and dried over Na₂SO₄. The organic extract was concentrated to give 35.

### Example 5 ― Synthesis of Meayamyacin Analogues

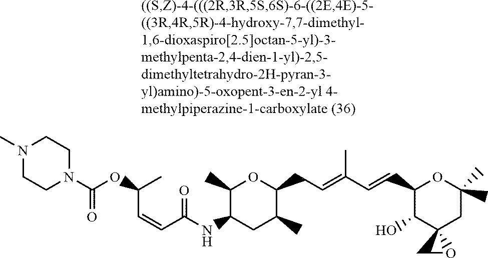

A solution of 35 (19 mg, 0.04 mmol, 1.0 equiv) in CH₂Cl₂ (0.8 mL) was treated with CDl (9.8 mg, 0.06 mmol, 1.5 equiv) and DMAP (1.0 mg, 0.008 mmol, 0.2 equiv). After 2 h, N-methylpiperazine (18 µL, 0.16 mmol, 5.0 equiv) was added and the mixture was stirred for an additional 4 h. After this time, the reaction was concentrated and the material purified by PTLC (SiO₂, 5% MeOH in CH₂Cl₂) to provide 36 (15.3 mg, 0.026 mmol, 65%) as an off white solid: ¹H NMR (600 MHz, CDCl₃) δ 6.39 (d, J = 15.6 Hz, 1H), 6.19 (dd, J = 11.9, 5.5 Hz, 1H), 6.02 - 5.83 (m, 3H), 5.72 (d, J = 12.2 Hz, 1H), 5.64 (dd, J = 15.7, 7.2 Hz, 1H), 5.54 (dd, J = 15.7, 7.7 Hz, 1H), 5.39 - 5.30 (m, 1H), 4.12 (d, J = 9.4 Hz, 1H), 3.94 (s, 1H), 3.70 - 3.60 (m, 2H), 3.53 (s, 5H), 3.37 - 3.32 (m, 1H), 2.38 - 2.26 (m, 5H), 2.22 (t, J = 7.8 Hz, 1H), 2.16 - 2.08 (m, 1H), 2.05 (d, J = 15.2 Hz, 1H), 1.98 ― 1.91 (m, 2H), 1.85 - 1.81 (m, 1H), 1.81 - 1.74 (m, 2H), 1.45 (s, 1H), 1.41 (d, J = 5.5 Hz, 1H), 1.36 - 1.32 (m, 4H), 1.31 (d, J = 4.4 Hz, 2H), 1.25 (s, 4H), 1.14 (d, J = 6.5 Hz, 3H), 1.02 (d, J = 6.9 Hz, 3H); HRMS-TOF-ESI (m/z) [M+H]⁺ calculated for C₃₂H₅₂N₃O₇ 590.3801, found 590.3805.

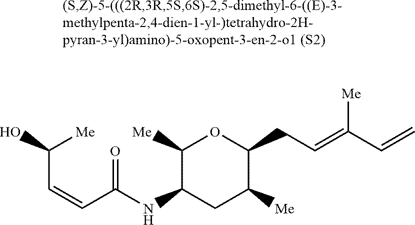

The alcohol S2 was prepared following a previously described procedure (Ref. 19) from 30 (K₂CO₃, MeOH, 0° C., 95%) and used crude to prepare the following derivatives without further characterization.

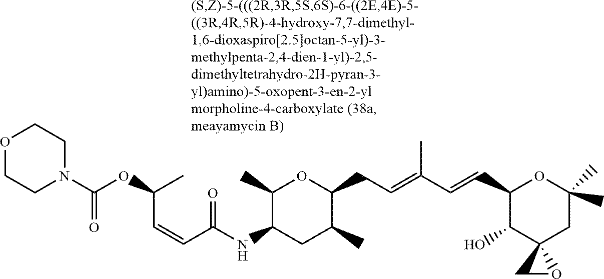

The alcohol S2 (15 mg, 0.05 mmol, 1.0 equiv) in CH₂Cl₂ (0.25 mL) was treated with CDI (24 mg, 0.15 mmol, 3.0 equiv) and the mixture was stirred for 12 h before the addition of morpholine (0.05 mL, 0.5 mmol, 10 equiv). After an additional 12 h, the reaction mixture was concentrated in vacuo. The crude material was purified by column chromatography (SiO₂, 50% EtOAc in hexanes) to give 37a (19 mg, 0.044 mmol, 86%) as a clear oil: ¹H NMR (600 MHz, CDCI₃) δ 6.36 (dd, J = 17.4, 10.7 Hz, 1H), 6.24 - 6.06 (m, 2H), 5.91 (dd, J = 11.6, 7.8 Hz, 1H), 5.70 (m, 1H), 5.45 (t, J = 7.2 Hz, 1H), 5.10 (m, 1H), 4.95 (m, 1H), 3.95 - 3.93 (m, 1H), 3.65 (s, 4H), 3.53 (td, J = 7.2, 2.7 Hz, 1H), 3.46 (s, 4H), 2.41 - 2.35 (m, 1H), 2.26 - 2.21 (dt, J = 15.0, 7.5 Hz, 1H), 1.96 - 1.93 (m, 2H), 1.75 (s, 3H), 1.40 (d, J = 6.5 Hz, 5H), 1.15 (d, J = 6.4 Hz, 4H), 1.02 (d, J = 7.4 Hz, 3H). ¹³C NMR (150 MHz, CDCI₃) J 165.1, 155.1, 144.3, 141.4, 135.8, 128.3, 122.4, 111.2, 80.9, 76.1, 70.3, 66.8, 47.2, 36.1, 32.0, 29.0, 22.9, 20.3, 18.0, 15.2, 12.1; IR (neat) vₘₐₓ 3354, 2926, 1690, 1668, 1458, 1276, 1242, 1168 cm⁻ ¹; HRMS-TOF-ESI (m/z) [M+H]⁺ calculated for C₂₃H₃₇NO₅S 421.2701, found 4421.2702.

A solution of 37a (11 mg, 0.026 mmol, 1.0 equiv) in CICH₂CH₂CI (0.25 mL) at 40° C. was treated with p-benzoquinone (0.8 mg, 0.008 mmol, 0.3 equiv), Grela cat.²⁵ (0.2 mL of 3.5 mg, 0.005 mmol, 0.2 equiv in 0.6 mL of CICH₂CH₂CI) and 7 (0.2 mL of 5.5 mg, 0.031 mmol, 1.2 equiv in 0.6 mL of CICH₂CH₂CI). The mixture was stirred for 1 h before a second portion of the solution of catalyst (0.2 mL) and 7 (0.2 mL) were added. After an additional 1 h, the final portion of catalyst (0.2 mL) and 7 (0.2 mL) were added. After 5 h, the reaction mixture was concentrated in vacuo. The crude material was purified by column chromatography (SiO₂, 80% EtOAc in hexanes) to give 38a (4.2 mg, 0.0072 mmol, 28%) as an off white solid: ¹H NMR (600 MHz, CD₂Cl₂) δ 6.38 (d, J = 15.8 Hz, 1H), 6.21 - 6.15 (m, 2H), 5.95 (dd, J = 11.6, 7.9 Hz, 1H), 5.91 (m, 1H), 5.74 (d, J = 11.6 Hz, 1H), 5.68 (dd, J = 15.7, 6.6 Hz, 1H), 5.56 (t, J = 7.6 Hz, 1H), 4.06 - 3.97 (m, 1H), 3.95 - 3.93 (m, 1H), 3.66 - 3.64 (m, 5H), 3.47 - 3.45 (m, 6H), 2.99 (d, J = 4.8 Hz, 1H), 2.50 - 2.49 (m, 1H), 2.40 (dd, J = 15.3, 7.6 Hz, 1H), 2.28 - 2.23 (dt, J = 15.4, 7.5 Hz, 1H), 2.20 (d, J = 14.3 Hz, 1H), 2.01 - 1.94 (m, 2H), 1.82 (m, 4H), 1.68 - 1.62 (m, 4H), 1.39 (s, 3H), 1.28 (s, 3H), 1.15 (d, J = 6.4 Hz, 3H), 1.06 (d, J = 7.4 Hz, 3H); IR (neat) vₘₐₓ 3415, 2973, 2926, 1689, 1669, 1639, 1520, 1427, 1242, 1115, 1059 cm⁻¹; HRMS-TOF-ESI (m/z) [M+H]⁺ calculated for C₃₁H₄₉N₂O₈, 577.3491 found 577.3489.

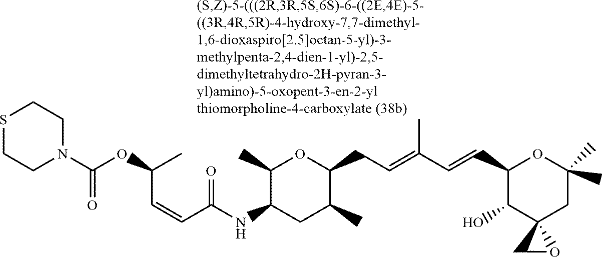

The alcohol S2 (15 mg, 0.049 mmol, 1.0 equiv) in CH₂CI₂ (0.25 mL) was treated with CDI (24 mg, 0.15 mmol, 3.0 equiv) and the mixture was stirred for 12 h before the addition of thiomorpholine (0.05 mL, 0.5 mmol, 10 equiv). After an additional 12 h, the reaction mixture was concentrated in vacuo. The crude material was purified by column chromatography (SiO₂, 50% EtOAc in hexanes) to give 37b (19 mg, 0.044 mmol, 70%) as a clear oil: ¹H NMR (600 MHz, CDCI₃) δ 6.36 (dd, J = 17.4, 10.7 Hz, 1H), 6.22 - 6.07 (m, 2H), 5.90 (dd, J = 11.6, 7.8 Hz, 2H), 5.69 (dd, J = 11.6, 1.4 Hz, 1H), 5.45 (t, J = 7.2 Hz, 1H), 5.10 (d, J = 17.4 Hz, 1H), 4.95 (d, J = 10.7 Hz, 1H), 4.00 -3.88 (m, 1H), 3.73 (d, J = 4.1 Hz, 4H), 3.67 (td, J = 6.4, 2.3 Hz, 1H), 3.53 (dt, J = 7.3, 3.6 Hz, 1H), 2.58 (d, J = 5.2 Hz, 7H), 2.49 - 2.29 (m, 1H), 2.24 (dt, J = 15.2, 7.6 Hz, 1H), 1.95 (tq, J = 9.4, 5.2, 4.8 Hz, 3H), 1.75 (s, 3H), 1.40 (d, J = 6.5 Hz, 5H), 1.15 (d, J = 6.4 Hz, 4H), 1.02 (d, J = 7.4 Hz, 3H); ¹³C NMR (150 MHz, CDCI₃) δ 165.1, 154.8, 144.3, 141.4, 135.8, 128.3, 122.4, 111.2, 80.9, 76.1, 70.3, 47.2, 36.0, 32.1, 29.0, 27.4, 20.4, 18.0, 15.2, 12.1; IR (neat) _{Vmax} 3349, 2927, 1688, 1667, 1521, 1422, 1223, 1049 cm⁻¹; HRMS-TOF-ESI (m/z) [M]⁺ calculated for C₂₃H₃₇NO₅S 437.2475, found 437.2474.

A solution of 37b (11 mg, 0.026 mmol, 1.0 equiv) in CICH₂CH₂CI (0.25 mL) at 40° C. was treated with p-benzoquinone (0.8 mg, 0.008 mmol, 0.3 equiv), Grela catalyst (Ref. 25) (0.2 mL of 3.5 mg, 0.005 mmol, 0.2 equiv in 0.6 mL of CICH₂CH₂CI) and 7 (0.2 mL of 5.5 mg, 0.031 mmol, 1.2 equiv in 0.6 mL of CICH₂CH₂CI). The mixture was stirred for 1 h before a second portion of the solution of catalyst (0.2 mL) and 7 (0.2 mL) were added. After an additional 1 h, the final portion of catalyst (0.2 mL) and 7 (0.2 mL) were added. After 5 h, the reaction mixture was concentrated in vacuo. The crude material was purified by column chromatography (SiO₂, 80% EtOAc in hexanes) to give 38b (2.2 mg, 0.0036 mmol, 14%) as an off white solid: ¹H NMR (600 MHz, CD₂Cl₂) δ 6.34 (d, J = 15.7 Hz, 1H), 6.19 - 6.07 (m, 2H), 5.91 (dd, J = 11.6, 7.8 Hz, 1H), 5.70 (dd, J = 11.7, 1.3 Hz, 1H), 5.64 (dd, J = 15.7, 6.6 Hz, 1H), 5.52 (t, J = 7.1 Hz, 1H), 3.96 (dd, J = 9.7, 6.6 Hz, 1H), 3.91-3.89 (m 1H), 3.77 - 3.66 (m, 6H), 3.66 (td, J = 6.4, 2.3 Hz, 1H), 3.54-3.52 (m, 1H), 3.48 (t, J = 9.9 Hz, 1H), 2.96 (d, J = 4.7 Hz, 1H), 2.57 (s, 4H), 2.46 (d, J = 4.7 Hz, 1H), 2.38-2.34 (m, 1H), 2.25 - 2.19 (m, 1H), 2.17 (d, J = 14.3 Hz, 1H), 1.96 - 1.91 (m, 1H), 1.78 (s, 3H), 1.36 (d, J = 1.6 Hz, 3H), 1.23 (s, 3H), 1.11 (d, J = 6.4 Hz, 3H), 1.02 (d, J = 7.3 Hz, 3H); IR (neat) _{Vmax} 3458, 2971, 2921, 1688, 1668, 1515, 1461, 1423, 1256, 1224, 1055, 971 cm⁻¹; HRMS-TOF-ESI (m/z) [M+H]⁺ calculated for C₃₁H₄₉N₂O₇S, 593.3262 found 593.3260.

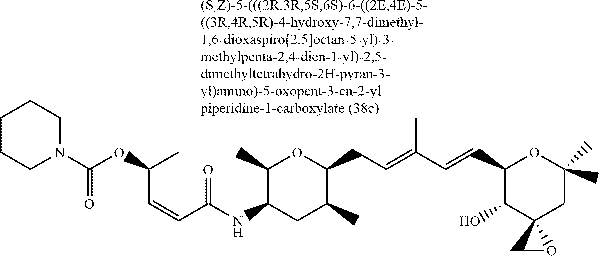

The alcohol S2 (12 mg, 0.040 mmol, 1.0 equiv) in CH₂CI₂ (0.25 mL) was treated with CDI (19 mg, 0.12 mmol, 3.0 equiv) and the mixture was stirred for 12 h before the addition of piperidine (0.05 mL, 0.4 mmol, 10 equiv). After an additional 12 h, the reaction mixture was concentrated in vacuo. The crude material was purified by column chromatography (SiO₂, 50% EtOAc in hexanes) to give 37c (7.5 mg, 0.018 mmol, 45%) as a clear oil; IR (neat) _{Vmax} 2919, 1703, 1667, 1469, 1235, 1091, 890 cm⁻¹; HRMS-TOF-ESI (m/z) [M+H]⁺ calculated for C₂₄H₃₉N₂O₄ 419.2910, found 419.2910.

A solution of 37c (7 mg, 0.017 mmol, 1.0 equiv) in CICH₂CH₂CI (0.25 mL) at 40° C. was treated with p-benzoquinone (0.5 mg, 0.005 mmol, 0.3 equiv), Grela cat.²⁵ (0.2 mL of 2.1 mg, 0.0034 mmol, 0.2 equiv in 0.6 mL of CICH₂CH₂CI) and 7 (0.2 mL of 3.6 mg, 0.02 mmol, 1.2 equiv. in 0.6 mL of CICH₂CH₂CI). The mixture was stirred for 1 h before a second portion of the solution of catalyst (0.2 mL) and 7 (0.2 mL) were added. After an additional 1 h, the final portions of catalyst (0.2 mL) and 7 (0.2 mL) were added. After 5 h, the reaction mixture was concentrated in vacuo. The crude material was purified by column chromatography (SiO₂, 80% EtOAc in hexanes) to give 38c (1.5 mg, 0.0027 mmol, 16%) as an off white solid: ¹H NMR (600 MHz, CDC1₃) δ 6.39 (d, J = 15.7 Hz, 1H), 6.31 (d, J = 9.1 Hz, 1H), 6.10 - 6.00 (m, 1H), 5.92 - 5.89 (m, 2H), 5.77 (dd, J = 15.4, 8.0 Hz, 1H), 5.71 - 5.62 (m, 2H), 5.50 (t, J = 7.3 Hz, 1H), 4.15 (t, J = 8.6 Hz, 1H), 4.04 - 3.87 (m, 2H), 3.66 - 3.64 (m, 1H), 3.56 - 3.48 (m, 3H), 3.41 - 3.39 (m, 5H), 3.36 (d, J = 9.2 Hz, 1H), 3.01 (dd, J = 18.3, 4.7 Hz, 1H), 2.58 (d, J = 1.8 Hz, 1H), 2.50 (t, J = 4.5 Hz, 1H), 2.39 - 2.35 (m, 1H), 2.23 - 2.17 (m, 2H), 2.01 -1.91 (m, 3H), 1.78 (s, 4H), 1.51 (s, 2H), 1.43 (s, 3H), 1.26 (s, 3H), 1.15 (d, J = 6.4 Hz, 3H), 1.02 (d, J = 7.2 Hz, 3H); IR (neat) _{Vmax} 3413, 2959, 1688, 1668, 1441, 1260, 1057, 1023, 801 cm¹; HRMS-TOF-ESI (m/z) [M+H]⁺ calculated for C₃₂H₅₁N₂O₇ 575.3696, found 575.3696.

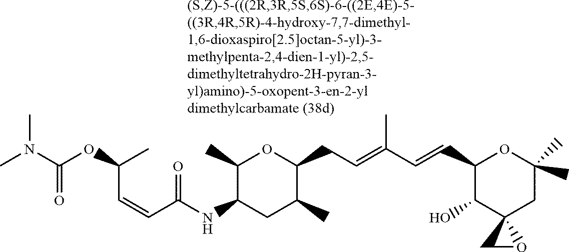

The alcohol S2 (10 mg, 0.033 mmol, 1.0 equiv) in CH₂CI₂ (0.25 mL) was treated with CDI (15.9 mg, 0.098 mmol, 3.0 equiv) and the mixture was stirred for 12 h before the addition of dimethylamine (0.012 mL, 0.16 mmol, 10 equiv). After an additional 12 h, the reaction mixture was concentrated in vacuo. The crude material was purified by column chromatography (SiO₂, 50% EtOAc in hexanes) to give 37d (5.6 mg, 0.015 mmol, 45%) as a clear oil: IR (neat) _{Vmax} 2919, 1705, 1666, 1468, 1389, 1235, 1091, 891 cm¹; HRMS-TOF-ESI (m/z) [M+H]⁺ calculated for C₂₁H₃₅N₂O₄, 379.2595 found 379.2597.

A solution of 37d (8 mg, 0.021 mmol, 1.0 equiv) in CICH₂CH₂CI (0.25 mL) at 40° C. was treated with p-benzoquinone (0.6 mg, 0.006 mmol, 0.3 equiv), Grela catalyst²⁵ (0.2 mL of 2.8 mg, 0.0042 mmol, 0.2 equiv in 0.6 mL of CICH₂CH₂CI) and 7 (0.2 mL of 4.5 mg, 0.025 mmol, 1.2 equiv in 0.6 mL of CICH₂CH₂CI). The mixture was stirred for 1 h before a second portion of the solution of catalyst (0.2 mL) and 7 (0.2 mL) were added. After an additional 1 h, the final portions of catalyst (0.2 mL) and 7 (0.2 mL) were added. After 5 h, the reaction mixture was concentrated in vacuo. The crude material was purified by column chromatography (SiO₂, 80% EtOAc in hexanes) to give 38d as an off white solid: ¹H NMR (600 MHz, CDCI₃) δ 6.39 (d, J = 15.7 Hz, 1H), 6.27 (d, J = 9.1 Hz, 1H), 6.13 - 5.99 (m, 1H), 5.91 (dd, J = 11.6, 7.8 Hz, 1H), 5.79 - 5.60 (m, 2H), 5.50 (t, J = 7.2 Hz, 1H), 4.13 - 3.99 (m, 1H), 3.94 (m, 1H), 3.65 (m, 1H), 3.60 - 3.44 (m, 2H), 3.02 (d, J = 4.7 Hz, 1H), 2.90 (s, 6H), 2.49 (d, J = 4.7 Hz, 1H), 2.40 - 2.34 (m, 1H), 2.27 - 2.09 (m, 2H), 2.01-1.87 (m, 2H), 1.78 (s, 3H), 1.60 (d, J = 23.6 Hz, 6H), 1.46 - 1.33 (m, 3H), 1.27 (d, J = 18.9 Hz, 3H), 1.15 (d, J = 6.5 Hz, 3H), 1.01 (d, J = 7.3 Hz, 3H); IR (neat) _{Vmax} 3359, 2925, 1686, 1668, 1521, 1441, 1260, 1057, 801 cm⁻¹; HRMS-TOF-ESI (m/z) [M+H]⁺ calculated for C₂₉H₄₇N₂O₇ 535.3383, found 535.3375.

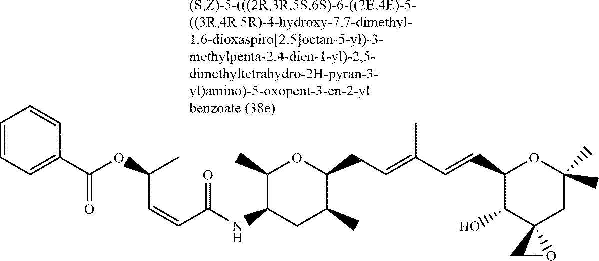

The alcohol S2¹⁹ (30 mg, 0.1 mmol, 1.0 equiv) in CH₂CI₂ (0.25 mL) was treated with benzoyl chloride (25.3 mg, 0.2 mmol, 2.0 equiv) and DMAP (36.7 mg, 0.3 mmol, 3 equiv). The mixture was stirred for 4 h, after which the reaction mixture was concentrated in vacuo. The crude material was purified by column chromatography (SiO₂, 30% EtOAc in hexanes) to give 37e (23 mg, 0.055 mmol, 55%): [α]²⁰D-46 (c 1.0, CHCl₃).

A solution of 37e (8 mg, 0.019 mmol, 1.0 equiv) in CICH₂CH₂CI (0.25 mL) at 40° C. was treated with p-benzoquinone (0.6 mg, 0.0057 mmol, 0.3 equiv), Grela catalyst²⁵ (0.2 mL of 3.8 mg, 0.0057 mmol, 0.3 equiv in 0.6 mL of CICH₂CH₂CI) and 7 (0.2 mL of 5.0 mg, 0.028 mmol, 1.5 equiv in 0.6 mL of CICH₂CH₂CI). The mixture was stirred for 1 h before a second portion of the solution of catalyst (0.2 mL) and 7 (0.2 mL) were added. After an additional 1 h, the final portions of catalyst (0.2 mL) and 7 (0.2 mL) were added. After 5 h, the reaction mixture was concentrated in vacuo. The crude material was purified by column chromatography (SiO₂, 80% EtOAc in hexanes) to give 38e (2.8 mg, 0.0049 mmol, 26%) as an off white solid: ¹H NMR (600 MHz, CDCI₃) δ8.06 - 8.03 (m, 2H), 7.57 - 7.53 (m, 1H), 7.43 (t, J = 7.8 Hz, 2H), 6.57 - 6.48 (m, 1H), 6.40 (dd, J = 16.0, 13.0 Hz, 1H), 6.04 (dd, J = 11.6, 7.7 Hz, 1H), 6.01 - 5.94 (m, 1H), 5.75 (dd, J = 11.5, 1.4 Hz, 1H), 5.59 - 5.47 (m, 1H), 5.45 - 5.27 (m, 1H), 4.19 - 4.05 (m, 1H), 4.03 - 3.93 (m, 2H), 3.70 - 3.64 (m, 1H), 3.58 - 3.49 (m, 2H), 3.03 (dd, J = 4.7, 1.1 Hz, 1H), 2.50 (d, J = 4.7 Hz, 1H), 2.45 - 2.33 (m, 1H), 2.23 - 2.18 (m, 1H), 2.03 - 1.92 (m, 2H), 1.78 (s, 3H), 1.54 (d, J = 6.5 Hz, 3H), 1.49 - 1.42 (m, 3H), 1.40 (d, J = 4.2 Hz, 3H), 1.28 (d, J = 3.4 Hz, 3H), 1.16 (dd, J = 6.5, 1.8 Hz, 3H), 1.03 (dt, J = 7.3, 4.8 Hz, 3H); HRMS-TOF-ESI (m/z) [M+H]⁺ calculated for C₃₃H₄₆NO₇ 568.3269, found 568.3277.

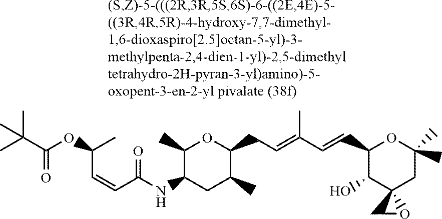

The alcohol S2¹⁹ (30 mg, 0.1 mmol, 1.0 equiv) in CH₂CI₂ (0.25 mL) was treated with pivaloyl chloride (24 mg, 0.2 mmol, 2.0 equiv) and DMAP (37 mg, 0.3 mmol, 3 equiv). The mixture was stirred for 4 h, after which the reaction mixture was concentrated in vacuo. The crude material was purified by column chromatography (SiO₂, 30% EtOAc in hexanes) to give 37f (19.5 mg, 0.050 mmol, 50%): [a]²⁰D -42.3 (c 1.0, CHCI₃).

A solution of 37f (18 mg, 0.046 mmol, 1.0 equiv) in CICH₂CH₂CI (0.25 mL) at 40° C. was treated with p-benzoquinone (1.45 mg, 0.014 mmol, 0.3 equiv), Grela catalyst²⁵ (0.2 mL of 9.2 mg, 0.014 mmol, 0.3 equiv in 0.6 mL of CICH₂CH₂CI) and 7 (0.2 mL of 12.1 mg, 0.068 mmol, 1.5 equiv in 0.6 mL of CICH₂CH₂CI). The mixture was stirred for 1 h before a second portion of the solution of catalyst (0.2 mL) and 7 (0.2 mL) were added. After an additional 1 h, the final portions of catalyst (0.2 mL) and 7 (0.2 mL) were added. After 5 h, the reaction mixture was concentrated in vacuo. The crude material was purified by column chromatography (SiO₂, 80% EtOAc in hexanes) to give 38f (4.5 mg, 0.0083 mmol, 18%) as an off white solid: ¹H NMR (600 MHz, CDCI₃) δ 9.79 (t, J = 2.1 Hz, 1H), 6.28 - 6.13 (m, 1H), 5.97 (dt, J = 27.7, 9.9 Hz, 1H), 5.88 (ddt, J = 10.9, 7.8, 2.6 Hz, 1H), 5.75 -5.66 (m, 1H), 5.09 - 4.81 (m, 1H), 4.37 - 4.23 (m, 1H), 4.19 - 4.06 (m, 1H), 4.06 - 3.90 (m, 1H), 3.83 - 3.62 (m, 2H), 3.62 - 3.44 (m, 1H), 3.05 (d, J = 4.7 Hz, 1H), 2.91 (d, J = 6.7 Hz, 1H), 2.68 (ddd, J = 16.5, 9.3, 2.5 Hz, 1H), 2.52 (d, J = 4.7 Hz, 1H), 2.39 (ddd, J = 16.4, 4.0, 1.8 Hz, 1H), 2.35 - 2.17 (m, 2H), 2.14 - 2.06 (m, 1H), 2.05 - 1.94 (m, 3H), 1.83 - 1.73 (m, 3H), 1.58 - 1.47 (m, 2H), 1.44 (dd, J = 13.7, 5.2 Hz, 1H), 1.41 (d, J = 6.5 Hz, 3H), 1.29 - 1.24 (m, 3H), 1.21 (d, J = 1.5 Hz, 9H), 1.18 - 1.15 (m, 2H), 1.05 (dtd, J = 14.8, 7.4, 6.3, 3.2 Hz, 3H); HRMS-TOF-ESI (m/z) [M+H]⁺ calculated for C₃₁H₅₀NO₇ 548.3582, found 548.3562.

### Example 6 - Alternative Order for the Subunit Assemblage

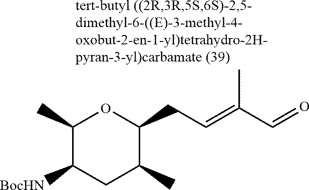

A solution of 15 (200 mg, 1.18 mmol, 1.0 equiv) in degassed CH₂Cl₂ (3.7 mL) was treated with methacrolein (0.98 mL, 11.8 mmol, 10 equiv) and Grubbs 2^{nd} generation catalyst²⁴ (98 mg, 0.12 mmol, 0.1 equiv) and the reaction mixture was stirred for 46 h. The volatiles were removed in vacuo and crude material was purified by column chromatography (SiO₂, 40% EtOAc in hexanes) to give 39 (261- mg, 0.84 mmol, 71%) as an off white solid:

$${\left[α\right]}_{D}^{20} - 28.4 \left(c 1.0 , {\text{CHCl}}_{3}\right) ;$$

¹H NMR (600 MHz, CDCl₃) δ9.40 (s, 1H), 6.52 (ddd, J = 7.7, 6.2, 1.4 Hz, 1H), 4.71 (d, J = 9.6 Hz, 1H), 3.63 - 3.60 (m, 2H), 3.59 - 3.56 (m, 1H), 2.56 - 2.51 (m, 1H), 2.38 (ddd, J = 15.7, 7.8, 5.2 Hz, 1H), 1.98 - 1.88 (m, 2H), 1.79 - 1.76 (m, 1H), 1.74 (s, 3H), 1.42 (s, 9H), 1.14 (d, J = 6.4 Hz, 3H), 1.05 (d, J = 7.4 Hz, 3H); ¹³C NMR (150 MHz, CDCI₃) δ195.2, 155.9, 150.7, 140.6, 79.8, 79.2, 76.6, 48.2, 36.0, 32.9, 29.7, 28.5, 17.7, 15.2, 9.6; IR (neat) _{Vmax}2975, 1710, 1685, 1495, 1364, 1235, 1165, 1061 cm⁻¹; HRMS-TOF-ESI (m/z) [M + Na]⁺ calculated for C₁₇H₂₉NO₄Na 334.1994, found 334.1987.

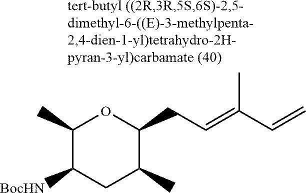

A solution of methyltriphenylphosphonium bromide (687 mg, 1.92 mmol, 2.0 equiv) in THF (1.3 mL) was treated with 1 M KO^{t}Bu in THF (1.73 mL, 1.73 mmol, 1.8 equiv) at 0° C. and stirred for 1 h. The aldehyde 39 (297 mg, 0.962 mmol) in THF (1 mL) was added dropwise. The reaction mixture was stirred for 12 h at 23° C., before being quenched with the addition of saturated aqueous NH₄Cl. After separation of the organic layer, the aqueous layer was extracted three times with EtOAc. The combined organic layer was washed with saturated aqueous NaCl, dried over Na₂SO₄, filtered and concentrated under reduced pressure. Flash chromatography (SiO₂, 10-40% EtOAc in hexane) provided 40 (263 mg, 0.87 mmol, 92%) as colorless oil: ¹H NMR (600 MHz, CDCl₃) δ 6.36 (dd, J = 17.4, 10.7 Hz, 1H), 5.45 (t, J = 7.3 Hz, 1H), 5.10 (d, J = 17.4 Hz, 1H), 4.94 (d, J = 10.7 Hz, 1H), 4.75 (d, J = 9.6 Hz, 1H), 3.60 (qd, J = 6.4, 2.2 Hz, 1H), 3.56 (ddd, J = 9.6, 4.3, 2.2 Hz, 1H), 3.50 (td, J = 7.3, 2.8 Hz, 1H), 2.37 (dt, J = 14.3, 6.9 Hz, 1H), 2.23 (dt, J = 15.2, 7.6 Hz, 1H), 1.96 - 1.86 (m, 3H), 1.75 (s, 3H), 1.44 (s, 9H), 1.14 (d, J = 6.4 Hz, 3H), 1.02 (d, J = 7.4 Hz, 3H); ¹³C NMR (150 MHz, CDCl₃) δ 156.0, 141.5, 135.7, 128.4, 111.2, 80.8, 79.1, 76.5, 48.5, 36.2, 32.1, 29.0, 28.6, 17.9, 15.1, 12.1; IR (neat) vₘₐₓ 1714, 1493, 1364, 1166, 1060 cm⁻¹; HRMS-TOF-ESI (m/z) [M + Na]⁺ calculated for C₁₈H₃₂NO₃ 310.2382, found 310.2373.

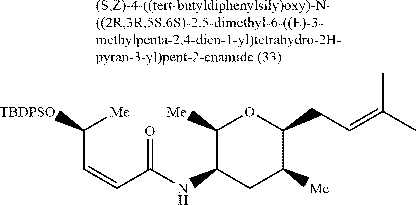

From 40: A sample of 40 (11.8 mg, 0.038 mmol, 1 equiv) was treated with 1 N HCI in EtOAc (1 mL). The solution was stirred at 23° C. for 15 min, after which the reaction mixture was concentrated in vacuo. A solution 27 (16 mg, 0.046 mmol, 1.2 equiv) in CH₃CN (1 mL) was treated with i-Pr₂NEt (26 µL, 0.152 mmol, 4.0 equiv) and O-(7-azabenzotriazol-1-yl)-N,N,N′,N′-tetramethyluronium hexafluorophoshate (HATU, 17 mg, 0.046 mmol, 1.2 equiv) at 0° C. The resulting mixture was stirred for 1 h at 0° C. before being added to the solution of the liberated free amine in CH₃CN (1 mL). The reaction mixture was stirred for 15 h at 23° C., before being quenched with the addition of saturated aqueous NH₄Cl. After separation of the organic layer, the aqueous layer was extracted three times with EtOAc. The combined organic layer was washed with saturated aqueous NaHCO₃ and saturated aqueous NaCl, dried over Na₂SO₄, filtered and concentrated under reduced pressure. Flash chromatography (SiO₂, 5-15% EtOAc in hexane) provided 33 (16.6 mg, 80%) as a colorless oil. Spectral data of this material were identical in all aspects to material prepared from 32.

### REFERENCES

Each of these references is incorporated herein by reference for all purposes.
(1)
   (a) Nakajima, H.; Sato, B.; Fujita, T.; Takase, S.; Terano, H.; Okuhara, M. New antitumor substances, FR901463, FR901464 and FR901465. 1. Taxonomy, fermentation, isolation, physicochemical properties and biological activities. J. Antibiot. 1996, 49, 1196-1203.
   (b) Nakajima, H.; Hori, Y.; Terano, H.; Okuhara, M.; Manda, T.; Matsumoto, S.; Shimomura, K. New antitumor substances, FR901463, FR901464 and FR901465. II. Activities against experimental tumors in mice and mechanism of action. J. Antibiot. 1996, 49, 1204-1211.
   (c) Nakajima, H.; Takase, S.; Terano, H.; Tanaka, H. New antitumor substances, FR901463, FR901464 and FR901465. III. Structures of FR901463, FR901464 and FR901465. J. Antibiot. 1997, 50, 96-99.
(2)
   (a) Liu, X.; Biswas, S.; Tang, G. L.; Cheng, Y. Q. Isolation and characterization of spliceostatin B, a new analogue of FR901464, from Pseudomonas sp. No. 2663. J. Antibiot. 2013, 66, 555-558.
   (b) He, H.; Ratnayake, A. S.; Janso, J. E.; He, M.; Yang, H. Y.; Loganzo, F.; Shor, B.; O’Donnell, C. J.; Koehn, F. E. Cytotoxic spliceostatins from Burkholderia sp. and their semisynthetic analogues. J. Nat. Prod. 2014, 77, 1864-1870.
(3) Liu, X.; Biswas, S.; Berg, M. G.;Antapli, C. M.; Xie, F.; Wang, Q.; Tang, M.-C.; Tang, G.-L.; Zhang, L.; Dreyfuss, G.; Cheng, Y.-Q. Genomics-guided discovery of thailanstatins A, B and C as pre-mRNA splicing inhibitors and anti-proliferative agents from Burkholderia thailandensis MSMB43. J. Nat. Prod. 2013, 76, 685-693.
(4)
   (a) Thompson, C. F.; Jamison, T. F.; Jacobsen, E. N. Total synthesis of FR901464. Convergent assembly of chiral components prepared by asymmetric catalysis. J. Am. Chem. Soc. 2000, 122, 10482-10483.
   (b) Thompson, C. F.; Jamison, T. F.; Jacobsen, E. N. FR901464: Total synthesis, proof of structure, and evaluation of synthetic analogues. J. Am. Chem. Soc. 2001, 123, 9974-9983.
(5)
   (a) Horigome, M.; Motoyoshi, H.; Watanabe, H.; Kitahara, T. A synthesis of FR901464. Tetrahedron Lett. 2001, 42, 8207-8210.
   (b) Motoyoshi, H.; Horigome, M.; Watanabe, H.; Kitahara, T. Total synthesis of FR901464: Second generation. Tetrahedron 2006, 62, 1378-1389.
(6)
   (a) Albert, B. J.; Sivaramakrishnan, A.; Naka, T.; Koide, K. Total synthesis of FR901464, an antitumor agent that regulates the transcription of oncogenes and tumor suppressor genes. J. Am. Chem. Soc. 2006, 128, 2792-2793.
   (b) Albert, B. J.; Sivaramakrishnan, A.; Naka, T.; Czaicki, N. L.; Koide, K. Total syntheses, fragmentation studies, and antitumor/antiproliferative activities of FR901464 and its low picomolar analogue. J. Am. Chem. Soc. 2007, 129, 2648-2659.
(7)
   (a) Ghosh, A. K.; Chen, Z.-H. Enantioselective syntheses of FR901464 and spliceostatin A: Potent inhibitors of spliceosome. Org. Lett. 2013,15, 5088-5091.
   (b) Ghosh, A. K.; Chen, Z.-H.; Effenberger, K. A.; Jurica, M. S. Enantioselective total syntheses of FR901464 and spliceostatin A and evaluation of splicing activity of key derivatives. J. Org. Chem. 2014, 79, 5697-5709.
(8) Review: Koide, K.; Albert, B. J. Total syntheses of FR901464. J. Synth. Org. Chem., Jpn. 2007, 65, 119-126.
(9)
   (a) Ghosh, A. K.; Veitschegger, A. M.; Nie, S.; Relitti, N.; MacRae, A. J.; Jurica, M. S. Enantioselective synthesis of thailanstatin A methyl ester and evaluation of in vitro splicing activity. J. Org. Chem. 2018, 83, 5187-5198.
   (b) Ghosh, A. K.; Reddy, G. C.; MacRae, A. J.; Jurica, M. S. Enantioselective synthesis of spliceostatin G and evaluation of bioactivity of spliceostatin G and its methyl ester. Org. Lett. 2018, 20, 96-99.
(10) Nicolaou, K. C.; Rhoades, D.; Kuma, S. M. Total syntheses of thailanstatins A-C, spliceostatin D, and analogues thereof. Stereodivergent synthesis of tetrasubstituted dihydro- and tetrahydropyrans and design, synthesis, biological evaluation, and discovery of potent antitumor agents. J. Am. Chem. Soc. 2018, 140, 8303-8320.
(11)
   (a) Lagisetti, C.; Pourpak, A.; Goronga, T.; Jiang, Q.; Cui, X.; Hyle, J.; Lahti, J. M.; Morris, S. W.; Webb, T. R. Synthetic mRNA splicing modulator compounds with in vivo antitumor activity. J. Med. Chem. 2009, 52, 6979-6990.
   (b) Lagisetti, C.; Palacios, G.; Goronga, T.; Freeman, B.; Caufield, W.; Webb, T. R. Optimization of antitumor modulators of pre-mRNA splicing. J. Med. Chem. 2013, 56, 10033-10044.
   (c) Makowski, K.; Vigevani, L.; Albericio, F.; Valcarcel, J.; Alvarez, M. Sudemycin K: A synthetic antitumor splicing inhibitor variant with improved activity and versatile chemistry. ACS Chem. Biol. 2017, 12, 163-173.
(12) Osman, S.; Albert, B. J.; Wang, Y.; Li, M.; Czaicki, N. L.; Koide, K. Structural requirements for the antiproliferative activity of pre-mRNA splicing inhibitor FR901464. Chem. - Eur. J. 2011, 17, 895-904.
(13) Ghosh, A. K.; Reddy, G. C.; Kovela. S.; Relitti, N.; Urabe, V. K.; Prichard, B. E.; Jurica, M. S. Enantioselective synthesis of a cyclopropane derivative of spliceostatin A and evaluation of bioactivity. Org. Lett. 2018, 20, 7293-7297.
(14)
   (a) Kaida, D.; Motoyoshi, H.; Tashiro, E; Nojima, T.; Hagiwara, M.; Ishigami, K.; Watanabe, H.; Kitahara, T.; Yoshida, T.; Nakajima, H.; Tani, T.; Horinouchi, S.; Yoshida, M. Spliceostatin A targets SF3b and inhibits both splicing and nuclear retention of pre-mRNA. Nat. Chem. Biol. 2007, 3, 576-578.
   (b) Lo, C.-W.; Kaida, D.; Nishimura, S.; Matsuyama, A.; Yashiroda, Y.; Taoka, H.; Ishigami, K.; Watanabe, H.; Nakajima, H.; Tani, T.; Horinouchi, S.; Yoshida, M. Inhibition of splicing and nuclear retention of pre-mRNA by spliceostatin A in fission yeast. Biochem. Biophys. Res. Commun. 2007, 364, 573-577.
(15) Albert, B. J.; McPherson, P. A.; O’Brien, K.; Czaicki, N. L.; DeStefino, V.; Osman, S.; Li, M.; Billy W. Day, B. W.; Grabowski, P. J.; Moore, M. M.; Vogt, A.; Koide, K. Meayamycin inhibits pre-mRNA splicing and exhibits picomolar activity against multidrug resistant cells. Mol. Cancer Ther. 2009, 8, 2308-2318.
(16)
   (a) Bonnal, S.; Vigevani, L.; Valcarcel, J. The spliceosome as a target of novel antitumour drugs. Nat. Rev. Drug Discovery 2012, 11, 847-859.
   (b) Desterro, J.; Bak-Gordon, P.; Carmo-Fonseca, M. Targeting mRNA processing as an anticancer strategy. Nat. Rev. Drug Discovery 2019, in press.
(17)
   (a) Koide et al., US 7,825,267 B2 (2010).
   (b) Osman, Waud, gorman, Day and Koide, Evaluation of FR901464 analogues in vitro and in vivo. Med. Chem. Commun. 2011, 2, 38.
(18)
   (a) Smith, A.B., III; Han, Q,; Breslin, P. A. S.; Beauchamp, G. K. Synthesis and Assignment of Absolute Configuration of (-)-Oleocanthol: A Potent, Naturally Occurring Non-Steroidal Anti-inflammatory and Anti-oxidant Agent Derived from Extra Virgin Olive Oils. Org. Lett. 2005, 7, 5075-5078.
   (b) Prabhakar, P.; Rajaram. S.; Reddy, D. K.; Shekar, V.; Venkateswarlu, Y. Total Synthesis of the phytotoxic stagonolides A and B. Tetrahedron:Asymmetry 2010, 21, 216-221.
(19) Koide, K.; Osman, S. Synthesis of FR901464 and analogs with antitumor activity. US Pat. App. 2015, US 20150307512A1.
(20) Wilson, Z. E.; Fenner, S.; Ley, S. V. Total syntheses of linear polythiazole/oxazole plantazolicin A and its biosynthetic precursor plantazolicin B. Angew. Chem. Int. Ed. 2015, 54, 1284-1288.
(21) Akwaboah, D. C.; Wu, D.; Forsyth, C. Stereoselective synthesis of the C1-C9 and C11-C25 fragments of amphidinolides C, C2, C3, and F. Org. Lett. 2017, 19, 1180-1183.
(22) Boger, D. L.; Brotherton, C. E. Total synthesis of azafluoranthene alkaloids: Rufescine and imeluteine. J. Org. Chem. 1984T, 49, 4050-4055.
(23)
   (a) Massad, S. K.; Hawkins, L. D.; Baker, D. C. A series of (2S)-2-O-protected-2-hydroxypropanals (L-lactaldehydes) suitable for use as optically active intermediates. J. Org. Chem. 1983, 48, 5180-5182.
   (b) Annunziata, R.; Cinquini, M.; Cozzi, F.; Dondio, G.; Raimondi, L. Intramolecular nitrile oxide cycloaddition on chiral olefins: A stereocontrolled approach to β-ketol precursors. Tetrahedron 1987, 43, 2369-2380.
   (c) Crimmins, M. T.; Jacobs, D. L. Asymmetric total synthesis of pyranicin. Org. Lett. 2009, 11, 2695-2698.
   (d) Srinivas, B.; Sridhar, R.; Rao, K. R. Stereoselective total synthesis of (+)-varitriol. Tetrahedron 2010, 66, 8527-8535.
(24) Scholl, M.; Ding, S.; Lee, C. W.; Grubbs, R. H. Synthesis and activity of a new generation of ruthenium-based olefin metathesis catalysts coordinated with 1,3-dimesityl-4,5-dihydroimidazol-2-ylidene ligands. Org. Lett. 1999, 1, 953-956.
(25)
   (a) Grela, K.; Harutyunyan, S.; Michrowska, A. A highly efficient ruthenium catalyst for metathesis reactions. Angew. Chem., Int. Ed. 2002, 41, 4038-4040.
   (b) Michrowska, A.; Bujok, R.; Harutyunyan, S.; Sashuk, V.; Dolgonos, G.; Grela, K. Nitro-substituted Hoveyda-Grubbs ruthenium carbenes: enhancement of catalyst activity through electronic activation. J. Am. Chem. Soc. 2004, 126, 9318-9325.
(26) Nicolaou, K. C.; Rhoades, D.; Lamani, M.; Pattanayak, Manas R.; Kumar, S. M. Total Synthesis of Thailanstatin A. J. Am. Chem. Soc. 2016, 138, 7532-7535.

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

1. A compound according to formula (I): 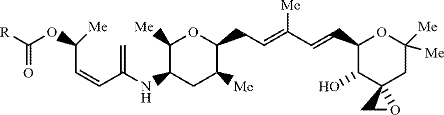
wherein R is 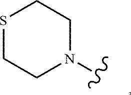 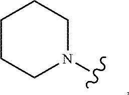 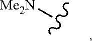 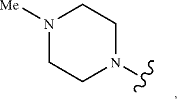 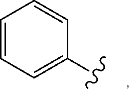
or 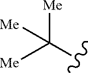
.

2. A compound according to claim 1, wherein R is 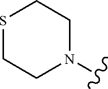
.

3. A compound according to claim 1, wherein R is 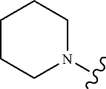
.

4. A compound according to claim 1, wherein R is 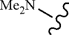
.

5. A compound according to claim 1, wherein R is 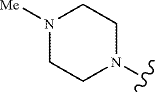
.

6. A compound according to claim 1, wherein R is 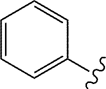
.

7. A compound according to claim 1, wherein R is 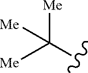
.

8. A method of treating a subject suffering cancer, comprising administering to such subject a therapeutically effective amount of a compound (I) according to claim 1.

9. A method according to claim 8, wherein the cancer is leukemia, colon cancer, or breast cancer.

10. A method according to claim 8, wherein, in compound (I), R is 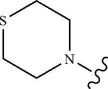
.

11. A method according to claim 8, wherein, in compound (I), R is 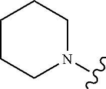
.

12. A method according to claim 8, wherein, in compound (I), R is 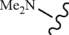
.

13. A method according to claim 8, wherein, in compound (I), R is 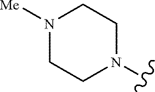
.

14. A method according to claim 8, wherein, in compound (I), R is 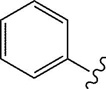
.

15. A method according to claim 8, wherein, in compound (I), R is 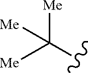
.
